(19) United States
(12) Patent Application Publication
(10) Pub. No.: US 2025/0263783 A1
(43) Pub. Date: Aug. 21, 2025

(54) VARIANT DETECTION METHODS AND COMPOSITIONS USING ARGONAUTE PROTEINS

(71) Applicant: 10x Genomics, Inc., Pleasanton, CA (US)

(72) Inventors: Hiroshi SASAKI, Dublin, CA (US); Peter SMIBERT, Danville, CA (US); Ruijie ZHANG, Milpitas, CA (US)

(21) Appl. No.: 19/059,173

(22) Filed: Feb. 20, 2025

Related U.S. Application Data
(60) Provisional application No. 63/556,352, filed on Feb. 21, 2024.

(51) Int. Cl. C12Q 1/6841; C12Q 1/6825; G01N 21/64

(52) U.S. Cl. CPC C12Q 1/6841; C12Q 1/6825; G01N 21/6428; C12Q 2600/156; G01N 2021/6439

(57) ABSTRACT

The present disclosure relates in some aspects to methods for analyzing target nucleic acids and their spatial locations in a biological sample. In some aspects, the presence/absence, amount, and/or identity of variant sequences (e.g., single nucleotide variations such as SNPs or point mutations) in a plurality of target nucleic acids in a cell or tissue sample are analyzed in situ in the sample. Also provided are compositions and kits for use in accordance with the methods.

### CROSS-REFERENCE TO RELATED APPLICATIONS

This application claims benefit of and priority to U.S. Provisional Application No. 63/556,352, filed on Feb. 21, 2024, entitled “Variant Detection Methods and Compositions Using Argonaute Proteins,” which is herein incorporated by reference in its entirety for all purposes.

### FIELD

The present disclosure relates in some aspects to methods and compositions for in situ analysis of nucleic acids and variant sequences therein in biological samples.

### BACKGROUND

Methods are available for detecting nucleic acids present in a biological sample. For instance, advances in single molecule fluorescent in situ hybridization (smFISH) have enabled nanoscale-resolution imaging of RNA in cells and tissues. However, analysis of short sequences (e.g., single nucleotide differences such as single nucleotide polymorphisms (SNPs) or point mutations) on individual transcripts in samples (e.g., a tissue section) has remained challenging. Improved methods for identifying short variant sequences and analyzing their spatial profiles (e.g., sequence identity, spatial location, and/or abundance) in cell or tissue samples are needed. Provided herein are methods, compositions, and kits that address such and other needs.

### BRIEF SUMMARY

Argonaute proteins are a large family of proteins that use nucleic acid guides to target other nucleic acids for binding and/or cutting. Family members are derived from prokaryotic and eukaryotic organisms. Some Argonaute family members use RNA guide nucleic acids, and some use DNA guide nucleic acids. The guide nucleic acid can direct Argonaute binding to a target sequence complementary to the guide nucleic acid or a portion thereof (e.g., complementary to a seed sequence of the guide nucleic acid) with high sensitivity. In the case of nuclease-active Argonaute proteins, the Argonaute and guide nucleic acid cut the complementary target nucleic acid sequence. Argonaute proteins include family members capable of binding and cutting RNA, as well as family members capable of binding and cutting DNA. Some Argonaute proteins lack nuclease activity. For such nuclease-deficient Argonaute proteins, the guide nucleic acid directs Argonaute binding to a specific nucleic acid sequence complementary to the guide nucleic acid sequence. In certain cases, Argonaute proteins are engineered to be nuclease-deficient.

The present application harnesses the sequence-specific binding or binding and cutting activities of Argonaute proteins for improved methods of in situ detection of variant sequences. In some aspects, the guide nucleic acid-mediated sequence-specific binding properties of nuclease-deficient Argonaute are used to provide improved methods of detecting a variant sequence in a rolling circle amplification product. In some embodiments, guide nucleic acid-mediated sequence-specific cutting activity of nuclease-active Argonaute is used to specifically degrade rolling circle amplification products comprising a specific sequence. In some aspects, this sequence-specific degradation of rolling circle amplification products (RCPs) has useful applications for discriminating between RCPs with different sequences (e.g., single nucleotide sequence differences). In other cases, the sequence-specific degradation of RCPs using Argonaute is used to remove undesired RCPs from a biological sample. In certain embodiments, highly abundant RCPs (such as RCPs generated from circularized probes or probe sets targeted to highly abundant mRNAs) are degraded, thereby reducing optical crowding for detecting remaining RCPs in the biological sample.

In some aspects, provided herein is a method for analyzing a biological sample, comprising: a) contacting the biological sample with a probe or probe set, wherein the probe or probe set comprises a first probe region and a second probe region that hybridize to a first target sequence and a second target sequence, respectively, in a target nucleic acid in the biological sample, wherein the first and second target sequences flank a gap sequence in the target nucleic acid, and wherein the gap sequence comprises a variant sequence; b) performing a gap-fill reaction on the probe or probe set to generate a gap-filled probe or probe set and circularizing the gap-filled probe or probe set; c) using a polymerase to amplify the circularized the gap-filled probe or probe set to generate a rolling circle amplification product (RCP) comprising multiple copies of the variant sequence in the biological sample; d) contacting the biological sample with a nuclease-deficient Argonaute protein and guide nucleic acid, wherein the guide nucleic acid comprises a sequence complementary to the variant sequence in the RCP, wherein the Argonaute protein and guide nucleic acid form a complex with the RCP; and e) detecting the complex formed between the Argonaute protein, the guide nucleic acid, and the RCP in the biological sample.

In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule.

In some embodiments, the Argonaute protein is a eukaryotic Argonaute protein. Alternatively, in some embodiments, the Argonaute protein is a prokaryotic Argonaute protein. In some embodiments, the Argonaute protein is a derivative or variant of a eukaryotic or prokaryotic Argonaute protein.

In some embodiments, the guide nucleic acid comprises a 5′-phosphate or a 5′-OH.

In some embodiments, the nuclease-deficient Argonaute protein is Ago1, Ago3, or Ago4. In some embodiments, the nuclease-deficient Argonaute protein is Ago1 or Ago4. In some embodiments, the nuclease-deficient Argonaute protein is a Drosophila Argonaute protein or a derivative or variant thereof. In some embodiments, the nuclease-deficient Argonaute protein is a nuclease-deficient Argonaute derived from Thermus thermophilus (dTtAgo). In some embodiments, the nuclease-deficient Argonaute protein comprises one or more inactivating mutations in a PIWI and/or PAZ domain of the Argonaute protein. In some embodiments, the guide nucleic acid and the Argonaute protein is bound in the complex before contacting the biological sample. In some embodiments, the guide nucleic acid and the Argonaute protein is contacted with the biological sample simultaneously.

In some embodiments, the sequence in guide nucleic acid complementary to the variant sequence is a seed sequence of the guide nucleic acid. In some embodiments, the sequence in guide nucleic acid complementary to the variant sequence comprises one or more nucleotides between positions 2 and 8 from the 5′ end of the guide nucleic acid.

In some embodiments, the nuclease-deficient Argonaute protein is labeled with a detectable moiety. In some embodiments, the detectable moiety is a fluorescent dye. In some embodiments, the guide nucleic acid is labeled with a detectable moiety. In some embodiments, the detectable moiety is a fluorescent dye.

In some embodiments, the guide nucleic acid comprises a 3′ tail sequence, and the method comprises contacting the biological sample with a detectably labeled probe that binds directly or indirectly to the 3′ tail sequence. In some embodiments, detecting the complex formed between the Argonaute protein, the guide nucleic acid, and the RCP in the biological sample comprises detecting the complex comprising the detectably labeled probe bound directly or indirectly to the guide nucleic acid.

In some embodiments, the detectably labeled probe is a first detectably labeled probe, and the method comprises sequential cycles of binding detectably labeled probes directly or indirectly to the 3′ tail sequence or sub-sequences thereof. In some embodiments, the biological sample is contacted with a plurality of different guide nucleic acids comprising seed sequences complementary to a plurality of different variant sequences.

In some embodiments, performing the gap-fill reaction comprises contacting the biological sample with a library of splint oligonucleotides, wherein each splint oligonucleotide comprises: i) ligatable ends; and ii) a hybridization region complementary to one of a plurality of different sequences, wherein a splint oligonucleotide of the library of splint oligonucleotides that is complementary to the gap sequence is ligated to the probe or probe set.

In some embodiments, the splint oligonucleotide comprises a 3′ hydroxyl group and a 5′ phosphate group. In some embodiments, the splint oligonucleotide comprises one or more ribonucleotide residues at and/or near its 3′ end and/or a 5′ flap configured to be cleaved by a structure-specific endonuclease. In some embodiments, the splint oligonucleotide and/or the gap sequence is between about 2 and about 40 nucleotides in length. In some embodiments, the variant sequence is at the 3′ or 5′ end of the gap sequence, and/or the sequence complementary to the variant sequence is at the 5′ or 3′ end of the splint oligonucleotide. In any of the embodiments herein, the variant sequence is at least 1, 2, 3, 4, 5, 6, 7, 8, 9, or more phosphodiester bonds from the 3′ or 5′ end of the gap sequence, and/or the sequence complementary to the variant sequence is at least 1, 2, 3, 4, 5, 6, 7, 8, 9, or more phosphodiester bonds from the 5′ or 3′ end of the splint oligonucleotide. In some embodiments, the variant sequence is at or near the central nucleotide(s) of the gap sequence and/or the sequence complementary to the variant sequence is at or near the central nucleotide(s) of the splint oligonucleotide.

In some embodiments, the variant sequence comprises a nucleotide variation, a nucleotide polymorphism, a mutation, a substitution, an insertion, a deletion, a translocation, a duplication, an inversion, and/or a repetitive sequence. In some embodiments, the variant sequence is two or more nucleotide residues in length. In some embodiments, the variant sequence is a single nucleotide. In some embodiments, the variant sequence is a single nucleotide variation (SNV), a single nucleotide polymorphism (SNP), a point mutation, a single nucleotide substitution, a single nucleotide insertion, or a single nucleotide deletion.

In some embodiments, the target nucleic acid is a target RNA. In some embodiments, the splint oligonucleotide is ligated to the probe or probe set using the target RNA as a template and a ligase having an RNA-templated DNA or RNA ligase activity. In some embodiments, the ligase is a Chlorella virus DNA ligase (PBCV DNA ligase) or a T4 RNA ligase. In some embodiments, the ligase is a PBCV-1 DNA ligase or a T4 RNA ligase 2.

In some embodiments, the library of splint oligonucleotides comprises at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different hybridization region sequences. In some embodiments, the molar concentration of the library of splint oligonucleotides is about equal to or about 2, about 4, about 8, about 10, or more times the molar concentration of the probe or probe set.

In some embodiments, the method comprises washing the biological sample after contacting with the library of splint oligonucleotides. In some embodiments, the washing is performed under less than stringent conditions.

In some embodiments, performing the gap-fill reaction comprises using an enzyme to extend an end of the probe or probe set using the target RNA as a template to generate an extended probe, wherein the extended probe is ligated to another end of the probe or probe set. In some embodiments, performing the gap-fill reaction comprises using a enzyme (e.g., a gap-fill polymerase) to extend an end of the probe or probe set using the target RNA as a template to generate an extended probe, wherein the extended probe is ligated to another end of the probe or probe set. In some embodiments, the gap-fill polymerase is a polymerase that has no or little strand displacement activity. In some embodiments, the gap-fill polymerase incorporates one or more deoxyribonucleotide residues and/or one or more ribonucleotide residues into a 3′ end of the probe or probe set to generate the extended probe. In some embodiments, the extended probe comprises one or more ribonucleotide residues at and/or near its 3′ end. In some embodiments, the extended probe is ligated to the probe or probe set using the target RNA as a template and a ligase having an RNA-templated DNA or RNA ligase activity. In some embodiments, the ligase is a Chlorella virus DNA ligase (PBCV DNA ligase) or a T4 RNA ligase. In some embodiments, the ligase is a PBCV-1 DNA ligase or a T4 RNA ligase 2.

In some embodiments, the polymerase to amplify the circularized the gap-filled probe or probe set is a Phi29 polymerase or a Bst polymerase.

In some embodiments, the variant sequence is among a plurality of different sequences. In some embodiments, the variant sequence is a mutant sequence or a minor variant among a plurality of different variant sequences. In some embodiments, the variant sequence is a wildtype sequence or a major variant among a plurality of different variant sequences. In some embodiments, the gap sequence comprises a genetic hotspot. In some embodiments, the gap sequence comprises two or more hotspot mutations.

In some embodiments, the guide nucleic acid comprises a guide sequence complementary to a sequence of the RCP comprising the variant sequence. In some embodiments, the guide sequence is between about 14 and 20 nucleotides in length. In some embodiments, the guide nucleic acid is between about 16 and 20 nucleotides in length.

In some embodiments, the first target sequence and the second target sequence are each about 15 to about 40 nucleotides in length. In some embodiments, the first target sequence and the second target sequence are each about 15 to about 20 nucleotides in length.

Also provided are methods comprising cleaving an RCP. In some aspects, provided herein is a method for analyzing a biological sample, comprising: a) contacting the biological sample comprising a rolling circle amplification product with an Argonaute protein and guide nucleic acid, wherein a complex comprising the Argonaute protein and the guide nucleic acid binds to a complementary sequence in the rolling circle amplification product (RCP) and cuts the complementary sequence in the RCP; b) washing the biological sample; and c) imaging the biological sample to detect the presence or absence of the rolling circle amplification product.

In some embodiments, the rolling circle amplification product is a rolling circle amplification product of a plurality of rolling circle amplification products, wherein at least one rolling circle amplification product of the plurality of rolling circle amplification products is cut by the Argonaute protein, and at least one rolling circle amplification product of the plurality of rolling circle amplification products is not cut by the Argonaute protein. In some embodiments, the rolling circle amplification product that is cut by the Argonaute protein comprises multiple copies of the complementary sequence. In some embodiments, the complementary sequence bound to the guide nucleic acid comprises an endogenous sequence in the biological sample or a complement thereof. In some embodiments, the rolling circle amplification product that is cut by the Argonaute protein is generated from a circular nucleic acid template that hybridizes to an RNA having high expression in the biological sample. In some embodiments, the RNA that has high expression is an RNA that is detected at a mean count of more than 20 transcripts per cell for at least a subset of cells in the biological sample.

In some embodiments, the method comprises imaging the biological sample to detect the at least one rolling circle amplification product of the plurality of rolling circle amplification products that is not cut by the Argonaute protein. In some embodiments, a sequence of the at least one rolling circle amplification product of the plurality of rolling circle amplification products that is not cut by the Argonaute protein is detected and/or analyzed at a location in the biological sample or a matrix embedding the biological sample. In some embodiments, the sequence of the at least one rolling circle amplification product of the plurality of rolling circle amplification products that is not cut by the Argonaute protein is detected and/or analyzed by sequential hybridization of detectably labeled probes, sequencing by hybridization, sequencing by ligation, sequencing by synthesis, sequencing by binding, sequencing by avidity, or a combination thereof.

In some embodiments, the complementary sequence in the RCP bound to the guide nucleic acid is 15-35 nucleotides in length. In some embodiments, the complementary sequence in the RCP bound to the guide nucleic acid is at least 22 nucleotides. In some embodiments, the guide nucleic acid comprises a seed region that binds the complementary sequence in the RCP. In some embodiments, the seed region comprises nucleotides 2-8 of the guide nucleic acid. In some embodiments, nucleotides 2-8 of the guide nucleic acid are fully complementary to the complementary sequence in the RCP.

In some embodiments, the RCP is generated from a probe or probe set, wherein the 3′ end and the 5′ end of the probe or probe set are ligated without gap filling prior to ligation. In some embodiments, the RCP is generated from a probe or probe set, wherein the ligation of a 3′ end and a 5′ end of the probe or probe set is preceded by a gap-fill reaction to generate a circularized nucleic acid template for rolling circle amplification. In some embodiments, the probe or probe set comprises a first probe region and a second probe region that hybridize to a first target sequence and a second target sequence, respectively, in a target nucleic acid in the biological sample, wherein the first and second target sequences flank a gap sequence in the target nucleic acid, wherein the gap sequence comprises a variant sequence, and wherein the variant sequence is bound by the complex comprising the Argonaute protein and the guide nucleic acid.

In some aspects, provided herein is a method for analyzing a biological sample, a) contacting the biological sample with a probe or probe set comprising a complement of barcode sequence, wherein the probe or probe set comprises a first probe region and a second probe region that hybridize to a first target sequence and a second target sequence, respectively, in a target nucleic acid in the biological sample, wherein the first and second target sequences flank a gap sequence in the target nucleic acid, and wherein the gap sequence comprises a region of interest comprising either a variant sequence of interest or an alternative sequence; b) performing a gap-fill reaction on the probe or probe set to generate a gap-filled probe or probe set; c) circularizing the gap-filled probe or probe set to generate a circularized probe comprising a gap-filled region complementary to the gap sequence; d) using a polymerase to amplify the circularized probe to generate a rolling circle amplification product (RCP) comprising multiple copies of the region of interest and the barcode sequence in the biological sample; e) contacting the biological sample with an Argonaute protein and guide nucleic acid, wherein a complex comprising the Argonaute protein and the guide nucleic acid binds to the alternative sequence of the region of interest and cuts the alternative sequence of interest; f) washing the biological sample; and g) imaging the biological sample to detect and/or analyze the presence or absence (or reduced presence) of the barcode sequence at the location in the biological sample, thereby analyzing the presence or absence of the variant sequence of interest.

In some aspects, provided herein is a method for analyzing a biological sample, comprising: a) contacting the biological sample with a probe or probe set comprising a complement of a barcode sequence, wherein the probe or probe set comprises a first probe region and a second probe region that hybridize to a first target sequence and a second target sequence, respectively, in a target nucleic acid in the biological sample, wherein the first and second target sequences flank a gap sequence in the target nucleic acid, and wherein the gap sequence comprises a variant sequence; b) performing a gap-fill reaction on the probe or probe set to generate a gap-filled probe or probe set; c) circularizing the gap-filled probe or probe set to generate a circularized probe comprising a gap-filled region complementary to the gap sequence; d) using a polymerase to amplify the circularized probe to generate a rolling circle amplification product (RCP) comprising multiple copies of the variant sequence and the barcode sequence in the biological sample; e) imaging the biological sample to detect the barcode sequence in the RCP at a location in the biological sample; f) contacting the biological sample with an Argonaute protein and guide nucleic acid, wherein a complex comprising the Argonaute protein and the guide nucleic acid binds to the variant sequence in the RCP and cuts the RCP; g) washing the biological sample; and h) imaging the biological sample to detect and/or analyze the presence or absence (or reduced presence) of the barcode sequence at the location in the biological sample.

In some embodiments, reduced presence of the barcode sequence or absence of the barcode sequence at the location in the biological sample in h) indicates the presence of the variant sequence at the location in the biological sample. In some embodiments, the presence of the barcode sequence at the location in the biological sample in h) indicates the absence of the variant sequence at the location in the biological sample.

In some embodiments, the method can comprise performing the gap-fill reaction, wherein performing the gap-fill reaction comprises contacting the biological sample with a library of splint oligonucleotides, wherein each splint oligonucleotide comprises: i) ligatable ends; and ii) a hybridization region complementary to one of a plurality of different sequences, wherein a splint oligonucleotide of the library of splint oligonucleotides that is complementary to the gap sequence is ligated to the probe or probe set.

In some embodiments, the splint oligonucleotide comprises a 3′ hydroxyl group and a 5′ phosphate group. In some embodiments, the splint oligonucleotide comprises one or more ribonucleotide residues at and/or near its 3′ end and/or a 5′ flap configured to be cleaved by a structure-specific endonuclease.

In some embodiments, the splint oligonucleotide and/or the gap sequence is between about 2 and about 40 nucleotides in length. In some embodiments, the variant sequence is at the 3′ or 5′ end of the gap sequence. In some embodiments, the sequence complementary to the variant sequence is at the 5′ or 3′ end of the splint oligonucleotide. In an some embodiments, the variant sequence is at least 1, 2, 3, 4, 5, 6, 7, 8, 9, or more phosphodiester bonds from the 3′ or 5′ end of the gap sequence. In some embodiments, the sequence complementary to the variant sequence is at least 1, 2, 3, 4, 5, 6, 7, 8, 9, or more phosphodiester bonds from the 5′ or 3′ end of the splint oligonucleotide. In some embodiments, the variant sequence is at or near the central nucleotide(s) of the gap sequence. In some embodiments, the sequence complementary to the variant sequence is at or near the central nucleotide(s) of the splint oligonucleotide.

In some embodiments, the variant sequence comprises a nucleotide variation, a nucleotide polymorphism, a mutation, a substitution, an insertion, a deletion, a translocation, a duplication, an inversion, and/or a repetitive sequence. In some embodiments, the variant sequence can comprises two or more nucleotide residues. In some embodiments, variant sequence is a single nucleotide. In some embodiments, the variant sequence is or comprises a single nucleotide variation (SNV), a single nucleotide polymorphism (SNP), a point mutation, a single nucleotide substitution, a single nucleotide insertion, or a single nucleotide deletion.

In some embodiments, the variant sequence is bound by the complex comprising the Argonaute protein and the guide nucleic acid.

In some embodiments herein, the Argonaute protein is an RNA-guided Argonaute, and the guide nucleic acid is an RNA molecule. In some embodiments, the Argonaute protein is a DNA-guided Argonaute, and the guide nucleic acid is a DNA molecule. In some embodiments, the Argonaute protein is a eukaryotic Argonaute protein. In some embodiments, the Argonaute protein is a prokaryotic Argonaute protein. In some embodiments, the Argonaute is a Thermus thermophilus Argonaute (TtAgo).

In some embodiments, the guide nucleic acid comprises a 5′-phosphate or a 5′-OH.

In some embodiments, circularizing the probe or probe set comprises: extending an end of the first probe by a polymerase using the target nucleic acid as a template to generate an extended circularizable probe, and circularizing the extended probe to generate a circularized probe. In some embodiments, the guide nucleic acid and the Argonaute protein are bound in a complex before contacting the biological sample. In some embodiments, the guide nucleic acid and the Argonaute protein are contacted with the biological sample simultaneously. In some embodiments, the Argonaute cleaves the RCP bound to the guide nucleic acid between any two positions between position 9 and 12 of the guide nucleic acid. In some embodiments, the Argonaute cleaves the RCP at a position between positions 10 and 11 of the guide nucleic acid.

In some embodiments, the method comprises incubating the biological sample with the Argonaute and the guide nucleic acid at a temperature between 20° C. and 50° C. to allow the cutting of the RCP by the Argonaute. In some embodiments, the temperature is between 30° C. and 44° C.

In some embodiments, the cutting of RCP by the Argonaute is performed in a buffer comprising Mg²⁺ and/or Mn²⁺. In some embodiments the cutting of the RCP by the RNA-cutting enzyme is performed in a buffer comprising Mg²⁺.

In some embodiments, the guide nucleic acid is between about 14 and 20 nucleotides in length. In some embodiments, the guide nucleic acid is between about 16 and 20 nucleotides in length. In some embodiments, the target nucleic acid is a cellular nucleic acid analyte or a product thereof. In some embodiments, the cellular nucleic acid analyte is an RNA and the product thereof is a cDNA. In some embodiments, the target nucleic acid is associated with a non-nucleic acid analyte. In some embodiments, the target nucleic acid is an oligonucleotide reporter in a labeling agent that binds to the analyte.

In some embodiments, the target nucleic acid is RNA. In some embodiments, the target nucleic acid is mRNA. In some embodiments, the target nucleic acid is a cDNA.

In some embodiments, the biological sample is a tissue section. In some embodiments, the biological sample is a cell or tissue sample. In some embodiments, the biological sample is a formalin-fixed, paraffin-embedded (FFPE) sample or a fresh frozen tissue sample. In some embodiments, the biological sample is fixed and/or permeabilized. In some embodiments, the biological sample is crosslinked and/or embedded in a matrix. In some embodiments, the matrix comprises a hydrogel. In some embodiments, the biological sample is cleared.

In some aspects, provided herein is a kit, comprising: a probe or probe set, wherein the probe or probe set comprises a first probe region and a second probe region that hybridize to a first target sequence and a second target sequence, respectively, in a target nucleic acid in the biological sample, wherein the first and second target sequences flank a gap sequence in the target nucleic acid, and wherein the gap sequence comprises a variant sequence; an Argonaute protein; and a guide nucleic acid capable of complexing with the Argonaute protein, wherein a seed region of the guide nucleic acid is complementary to the variant sequence. In some embodiments, the Argonaute is nuclease-deficient. In some embodiments, the Argonaute is nuclease active.

In some embodiments, provided herein is a system comprising a biological sample; a probe or probe set, wherein the probe or probe set comprises a first probe region and a second probe region that hybridize to a first target sequence and a second target sequence, respectively, in a target nucleic acid in the biological sample, wherein the first and second target sequences flank a gap sequence in the target nucleic acid, and wherein the gap sequence comprises a variant sequence; an Argonaute protein; and a guide nucleic acid capable of complexing with the Argonaute protein, wherein a seed region of the guide nucleic acid is complementary to the variant sequence or a complement thereof. In some embodiments, the system comprises a ligase for ligating the probe or probe set. In some embodiments, the system comprises a polymerase for performing a gap-fill reaction of the probe or probe set. In some embodiments, the system comprises a polymerase for performing a rolling circle amplification (RCA) reaction. In some embodiments, the system comprises a plurality of detectably labeled probes.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows scenarios in detecting single-nucleotide variants in target RNAs (e.g., of Allele X or Allele Y) using probe hybridization and ligation to discriminate among single nucleotides of interest. For instance, a padlock probe can hybridize to an RNA sequence such that an interrogatory nucleotide (for example, X′ which is complementary to nucleotide X, or Y′ which is complementary to nucleotide Y) is at a ligation junction. Incorrect detection of the variant sequence can result from the high error rate (e.g., ˜5%) of a ligase when the ligation is templated on RNA. This can result in false positive detection of the variant nucleotide as well as failure to detect a true positive signal associated with the variant nucleotide.

FIG. 2 shows an example method of detecting variant sequences using a probe or probe set that is gap-filled using a target nucleic acid (e.g., RNA or cDNA) as a template to generate a circularized probe and an RCP of the circularized probe, and subsequently using a detectably labeled Argonaute-guide nucleic acid complex to detect a variant sequence on the RCP.

FIG. 3A shows example probes or probe sets which are gap-filled using hybridization of a splint oligonucleotide to the probe or probe set (left panel) or polymerase extension followed by ligation of the extended probe or probe set (right panel). FIG. 3B shows a schematic of the Argonaute-guide nucleic acid complex, in which the seed region of the guide nucleic acid hybridizes to the RCP at the variant sequence.

FIG. 4A shows an example method of detecting the presence or absence of a variant sequence among a plurality of variant sequences at a location in the biological sample using a barcoded probe or probe set that is gap-filled using a target nucleic acid (e.g., RNA) as a template to generate a circularized probe and an RCP of the circularized probe, performing a first round of imaging to detect the barcode, using a slicer Argonaute-guide nucleic acid complex to cut the RCP at variant sequence, washing the sample to remove cut RCPs, performing a second round of imaging the sample to detect the barcode again, and comparing the first and second rounds of imaging to detect the presence or absence of the variant sequence at the location in the biological sample. FIG. 4B shows a decoding metric for detecting the presence or absence of a variant sequence across two rounds of imaging.

FIG. 5 shows an example method of detecting the presence or absence of a variant sequence X at a location in a biological sample using a barcoded probe or probe set that is gap-filled using a target nucleic acid (e.g., RNA) as a template to generate a circularized probe and an RCP of the circularized probe, using slicer Argonaute-mediated obliteration to cut alternative variant Y, washing the sample, then imaging the sample to detect the presence or absence of the barcode associated with variant X.

FIG. 6A and FIG. 6B provide a schematic illustration of Argonaute-mediated obliteration to reduce optical crowding during imaging. FIG. 6A shows an example of overlapping signals generated in situ during sequential rounds of detection steps, at a resolution that makes accurate detection of individual spot signals and barcodes at a location in the tissue sample challenging. FIG. 6B shows decoding of the same sample following Argonaute-mediated obliteration of a variant sequence, demonstrating improved optical resolution of the signal code with a lower density of detection molecules present at a location in the biological sample.

### DETAILED DESCRIPTION

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Single nucleotide variants (SNVs) are the most common genetic variants and ubiquitous in genomes and transcriptomes. The ability to spatially locate and quantify SNVs in cells and tissue samples has the potential to significantly propel our understanding of biology. Transcriptomic SNV detection with current technologies is challenging due to the biochemistry involved. Some in situ detection methods depend on the RNA-templated ligation of circularizable probes (e.g., padlock probes) using a ligase. However, options of RNA-templated ligases are more limited (compared to DNA-templated ligases) and ligases used for RNA-templated ligation often suffer from ligation junction biases as well as poor ability to discriminate between matched and mismatched nucleotides at the ligation junctions.

Argonaute proteins are a large family of proteins that use nucleic acid guides to target other nucleic acids and either bind or cut at a defined location in a target sequence in the target nucleic acid. Argonaute family members are derived from prokaryotic and eukaryotic organisms. Some Argonaute family members use RNA guides as guide nucleic acids. Some Argonaute family members use DNA guides as guide nucleic acids. Some Argonaute family members bind RNA. Some Argonaute family members bind and cut RNA. Some Argonaute family members bind, but do not cut, RNA. Some Argonaute family members bind DNA. Some Argonaute family members bind and cut DNA. Some Argonaute family members bind, but do not cut, DNA. Argonaute proteins that cut a target nucleic acid are said to have slicer activity. Not all Argonaute proteins have slicer activity; for example, Argonaute proteins involved in miRNA-mediated post-transcriptional regulation are slicer-dead (i.e., the Argonaute-guide nucleic acid binds, but does not cut, at the target sequence). While Argonaute proteins are endogenously involved in gene regulation and defense from pathogenic sequences, Argonaute proteins have been demonstrated to be useful tools for molecular biology.

Slicer-dead (i.e., catalytically inert or nuclease-dead) Argonaute proteins with guide complexes can compete for binding at SNPs with better success than hybridization of free oligonucleotides not in a complex with an Argonaute protein. Argonaute-guide nucleic acid complexes can hybridize with target sites faster than free oligonucleotides competing for the same target sites. Argonaute-guide nucleic acids also have a very low rate of off-target binding. This binding accuracy is due to the high sensitivity of the guide nucleic acid seed region (e.g., the seed region comprising nucleotides 2-8 at the 5′ end of the guide nucleic acid) to single-nucleotide mismatches. The guide nucleic acid requires full sequence complementarity to the target strand throughout the seed region. For some Argonaute proteins (e.g., such as non-cutting Argonaute proteins involved in regulation of miRNAs), sequence complementarity of a supplementary 3′ region with the nucleic acid target is also required for successful binding of the Argonaute-guide nucleic acid complex in addition to complementarity of the seed sequence.

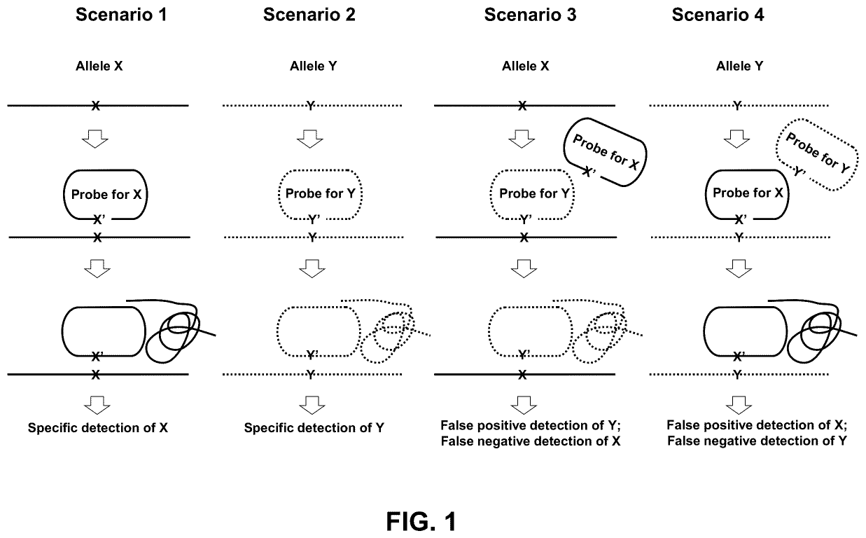

Certain methods for detecting variant sequences aim to discriminate a variant sequence (e.g., a particular SNP or point mutation) at the probe hybridization/ligation step. For instance, padlock probes with only one base difference in the arms (e.g., at the 3′ or 5′ end of the probes) targeting a single nucleotide difference in target nucleic acids can compete with each other for hybridization to a target sequence. In some embodiments, the best matching probe outcompetes the other probes, becomes more stably hybridized to a target molecule, and is circularized using a ligase and the target sequence as a ligation template. In some embodiments, the circularizable probe or probe set comprises a 5′ flap that does not hybridize to the target sequence, wherein the 5′ flap is configured to be cleaved by a structure-specific endonuclease. In certain embodiments, the ligated probe is amplified using rolling circle amplification (RCA) (e.g., as illustrated in Scenario 1 and Scenario 2 in FIG. 1) and detected by various readout means. For instance, a barcode sequence in the padlock probe that corresponds to the matching variant sequence (e.g., a point mutation or a wildtype nucleotide) can be detected using hybridization based methods to detect the barcode sequence and identify whether a rolling circle amplification product (RCP) is generated from a mutant or wildtype padlock probe.

Assays using probe hybridization/ligation to discriminate variant sequences can suffer from low specificity for multiple reasons, including properties of the ligase and/or the target nucleic acid. Low ligase fidelity can result in formation and detection of a ligation product (and subsequent amplicons of the ligation product), even when the sequence of interest does not match an interrogatory region of a probe, producing a high level of background or false positive results. For example, RNA templated ligases can tolerate some mismatches. In addition, ligases can have a strong base preference and probe end bias. Moreover, in the case of padlock probes targeting a single nucleotide difference, the single nucleotide is usually targeted by one arm, while the other arm covers a common region (e.g., a conserved region) among nucleic acid molecules containing different bases at the single nucleotide position of interest. As a result, on the one hand, one arm of a first padlock probe could hybridize to the common region in a particular target nucleic acid molecule, but the other arm of the first padlock probe does not fully match the single nucleotide position in the particular target nucleic acid molecule. Due to low ligase fidelity on RNA templates, the nonspecifically hybridized probes can nevertheless be ligated and amplified by RCA (e.g., as illustrated in Scenario 3 and Scenario 4 in FIG. 1). On the other hand, one arm of a second padlock probe could perfectly match the single nucleotide position in the target nucleic acid molecule, but the other arm of the second padlock probe cannot hybridize to the common region which is occupied by the first padlock probe or another probe. This is either ligated to a two-probe chimera that cannot be amplified by RCA, or probe hybridization can become unstable so that none of the padlock probes generates a ligation product and subsequently a detectable RCP, which can lead to a drop in detection efficiency. Improved methods for analyzing nucleic acids present in a biological sample, such as for detecting a single nucleotide of interest in situ are needed.

In some embodiments, provided herein are methods and compositions that reduce errors in detecting a variant nucleotide or short variant sequence (e.g., an SNV) using an Argonaute/guide nucleic acid complex to detect a variant nucleotide or short variant sequence (e.g., in a rolling circle amplification product). As shown in FIG. 1, in situ SNV detection can be hampered by low ligase specificity when ligation is templated on RNA targets, where high false ligation rates (e.g., >5%) when ligating DNA probes hybridized to RNA templates can be observed in Scenarios 3 and 4. In some embodiments, provided herein is an approach to capture a short variant nucleotide or sequence with a high level of specificity by taking a gap-fill approach using a probe or probe set (e.g., a padlock probe or split probe set) that hybridizes to a target RNA, amplifying the circularized gap-filled probe by RCA, and then performing a detection by using an Argonaute-guide nucleic acid complex with a seed region complementary to the short variant nucleotide or sequence to discriminate the variant nucleotide or short sequence which can be present in numerous copies in the RCP and in the form of DNA. In some embodiments, the seed sequence in the Argonaute-guide nucleic acid complex hybridized to the RCP is at the variant nucleotide or short sequence incorporated into the RCP via gap-filling and subsequent RCA. In some embodiments, an Argonaute-guide nucleic acid complex can comprise a detectable label and/or a detectable region, such that the Argonaute-guide nucleic acid complex can be detected when bound to a target nucleic acid in a biological sample (e.g., when bound to an RCP generated from a gap-filled circularized probe) by detecting the detectable label and/or the detectable region. In some embodiments, the Argonaute-guide nucleic acid complex does not comprise a detectable label (e.g., a fluorophore) and comprises a detectable region which directly or indirectly binds to a detectably labeled probe. In some embodiments, the detectable region comprises a detectably labeled oligonucleotide binding sequence or a barcode region. In some embodiments, in situ detection of the RCP comprising multiple copies of the variant sequence and in situ detection of the Argonaute-guide nucleic acid complex do not comprise using a base-by-base detection method, such as sequencing-by-synthesis (SBS), sequencing-by-ligation (SBL), sequencing-by-binding (SBB), or avidity sequencing, or a combination thereof. In some embodiments, copies of the variant sequence in the RCP are not sequenced using SBS, SBL, SBB, avidity sequencing, or a combination thereof.

In some embodiments, detection of a variant sequence with an Argonaute-guide nucleic acid complex is highly specific due to the requirement for exact sequence complementarity within all or a part of the seed region of the guide nucleic acid of the Argonaute-guide nucleic acid complex. In some embodiments, the seed region of the guide nucleic acid comprises 5′ nucleotides 2-8. In some embodiments, the seed region comprises one or more interrogatory nucleotides that are complementary to a variant sequence. In some embodiments, nucleotides 6 and/or 7 of the guide nucleic acid comprise interrogatory nucleotides. In some embodiments, most or all of the seed region of the guide nucleic acid must be complementary to the target nucleic acid in order for target recognition and binding of the Argonaute-guide nucleic acid complex to the target nucleic acid to occur.

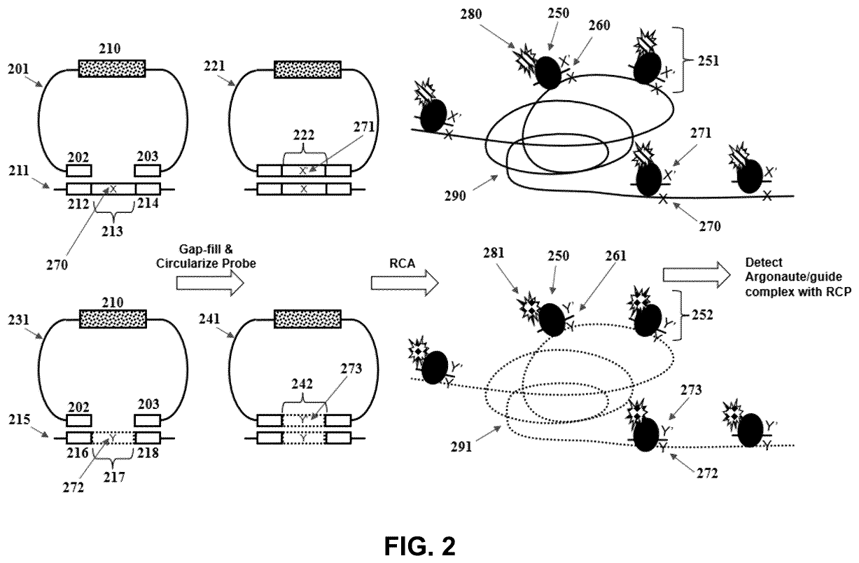

In some embodiments, provided herein are methods and compositions that reduce false positive signals (e.g., false positive signals due to incorrect probe ligation) for the identification of variant sequences in cell or tissue samples (e.g., SNPs) using in situ assays. In some embodiments, a method of SNV detection disclosed herein uses a probe or probe set which is a gap-fill probe or probe set, with the gap over the SNV site or a hotspot region containing the SNV site. In some embodiments, the gap in the probe or probe set is filled with an oligonucleotide (e.g., a splint oligonucleotide) or through polymerization using a reverse transcriptase, followed by a ligation step in order to generate a circularized probe. In some embodiments, rolling circle amplification is performed using the circularized probe, generating an RCP containing multiple copies of the SNV (or a hotspot region containing the SNV) and the RCPs corresponding to different SNVs are detected via the binding of a detectably labeled, slicer-dead Argonaute-guide nucleic acid complex to the variant sequence, as shown in FIG. 2.

In some embodiments, detection of the multiple copies of the SNV (or a hotspot region containing the SNV) in the RCP comprises using an Argonaute-guide nucleic acid complex that binds to the RCP at the variant sequence. In some embodiments, the guide nucleic acid comprises a barcode region with different barcode sequences that correspond to different variant sequences in a target nucleic acid, such as a first barcode region corresponding to a mutant SNV and a second barcode region corresponding to a wildtype SNV. In some embodiments, the barcode region of the guide nucleic acid is located in the optional 3′ tail region of the guide nucleic acid. In some embodiments, the Argonaute protein of the Argonaute-guide nucleic acid complex comprises a barcode region with different barcode sequences for different variant sequences in a target nucleic acid, such as a first barcode region corresponding to a mutant SNV and a second barcode region corresponding to a wildtype SNV, enabling decoding of the transcripts in a sample to determine the presence or absence of the mutant SNV and/or the wildtype SNV. In some embodiments, a first set of Argonaute-guide nucleic acid complexes with an Argonaute protein comprising a first barcode region has a first guide nucleic acid comprising a seed region complementary to a wildtype SNV, and a second set of Argonaute-guide nucleic acid complexes with an Argonaute protein comprising a second barcode region has a second guide nucleic acid comprising a seed region complementary to a mutant SNV. In some embodiments, the method comprises decoding of the Argonaute barcodes in a sample, thereby determining the presence or absence of the mutant SNV.

In some embodiments, a method disclosed herein does not rely on the discrimination of a variant sequence during the hybridization of the probe or probe set to target RNAs, or the discrimination of the variant sequence during RNA-templated ligation of the probe or probe set. In some embodiments, the method comprises variant sequence detection and identification (e.g., genotyping) in a readout stage, e.g., after the probe or probe set hybridization to a target RNA, gap-filling and circularization, and generation of RCPs. In some embodiments, the method comprises using binding of a seed sequence of a guide nucleic acid of a guide nucleic acid-Argonaute complex to an RCP. In some embodiments, the Argonaute-guide nucleic acid complex comprises a seed sequence which is configured to bind to the RCP and the molecule comprising an interrogatory nucleotide for the SNV is detectably labeled. In some embodiments, the guide nucleic acid of the Argonaute-guide nucleic acid complex comprises a seed sequence which is configured to bind to the variant sequence in the RCP. In some embodiments, the Argonaute-guide nucleic acid complex comprises a detectable label or a detectable region (e.g., the 3′ end of the guide nucleic acid comprises a detectable region). In some embodiments, the detectable region comprises a barcode region comprising one or more barcode sequences, and one or more detectably labeled probes complementary to the barcode sequence(s) are used to decode the barcode region. In some embodiments, the readout comprises identifying the sequence variations using sequential hybridization of probes (e.g., detectably labeled probes, or intermediate probes configured to directly or indirectly bind to detectably labeled probes) to the Argonaute-guide nucleic acid complex (e.g., at a barcode sequence in the guide nucleic acid).

### II. Detecting Variant Sequences in RCA Products with Argonaute-Guide Nucleic Acid Complexes

Argonaute-mediated hybridization of a guide nucleic acid to a target sequence may offer several advantages over RNA-templated DNA ligation for accurate detection and discrimination of variant sequences such as single nucleotide polymorphisms (SNPs). Argonaute-mediated hybridization of a guide nucleic acid to a target nucleic acid may occur more rapidly than probe hybridization in the absence of an Argonaute helper protein. Requirements for complementarity of the guide nucleic acid seed region and/or 3′ supplementary region may provide more stringent matching criteria than hybridization, allowing for precise detection and discrimination of single-nucleotide differences within these regions of the guide nucleic acid.

Argonaute proteins can be nuclease-active (i.e., have slicer activity) or nuclease-deficient (i.e., lack slicer activity). In some embodiments, provided herein is a method comprising contacting a biological sample with a nuclease-deficient Argonaute protein and a guide nucleic acid, wherein the nuclease-deficient Argonaute protein and the guide nucleic acid form a complex with a sequence complementary to the guide nucleic acid. In some embodiments, the nuclease-deficient Argonaute proteins comprise a detectable moiety such as a fluorescent label. In some embodiments, the guide nucleic acid is directly labeled with a detectable moiety. In some embodiments, the guide nucleic acid comprises a detectable moiety. In some embodiments, the guide nucleic acid is indirectly labeled with a detectable moiety. In some embodiments, the method comprises detecting the Argonaute/guide nucleic acid bound to a variant sequence at a location in the biological sample, thereby detecting the complementary sequence of the guide nucleic acid at the location in the biological sample.

In some embodiments, the complementary sequence of the guide nucleic acid is a variant sequence of interest (e.g., a single nucleotide variant or single nucleotide polymorphism). In some embodiments, a variant sequence present in a cellular nucleic acid is incorporated into a rolling circle amplification product using gap-fill circularizable probes or probe sets that are circularized and amplified to generate the rolling circle amplification product. In certain embodiments, a complement of the variant sequence is incorporated into the circularized probe in the gap-fill reaction, and the rolling circle amplification using the circularized probe as a template provides a rolling circle amplification product comprising multiple copies of the variant sequence. In some embodiments, the gap-fill probe or probe set and gap-fill reaction is according to any of the embodiments described in Section IV. In some embodiments, the rolling circle amplification is performed according to any of the embodiments described in Section V.

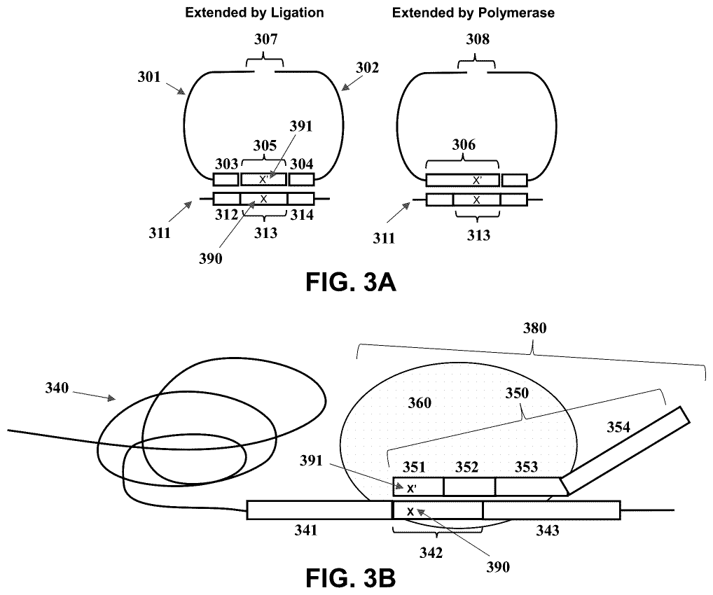

An embodiment of a method for detecting a variant sequence of interest at a location in a biological sample is shown in FIG. 2. This embodiment comprises using rolling circle amplification of a gap-fill padlock probe or probe set 201 targeting a variant sequence X 270, 272 Y, and then using optically labeled Argonaute-guide nucleic acid complexes 251, 252 to bind and detect a variant sequence of interest at a location in the rolling circle amplification products. A padlock probe or probe set 201 with an optional barcode region 210 comprises a first probe region 202 complementary to a first target sequence 212 in a target nucleic acid 211 and a second probe region 203 complementary to a second target sequence 214. The two target regions 202, 203 in the target nucleic acid 211, 215 flank a gap sequence 213 comprising a variant sequence. The variant sequence within the gap sequence of the target sequence may comprise either the variant of interest X 270 or an alternative variant Y 272 at a location in a target nucleic acid 211, 215. The probe or probe set 201, 231 hybridizes to the two target regions 212, 214 flanking the gap sequence 213, 217, and subsequently the padlock probe is gap-filled (via either splint oligonucleotide ligation or a polymerase, as shown in FIG. 3A left and right panels, respectively) to generate a circularized probe 221, 241 comprising a gap-filled region 222, 242. The gap-filled region comprises either a gap-fill region 222 comprising the complement sequence X′ 271 complementary to the variant sequence of interest X 270, or a gap-filled region 242 comprising an alternative complement sequence Y′ 273 complementary to the alternative variant sequence Y 272.

In some embodiments, the method presented herein further comprises contacting an RCP generated from a biological sample with an Argonaute-guide nucleic acid complex. In some embodiments, the guide nucleic acid and the Argonaute protein of the Argonaute-guide nucleic acid complex form a complex prior to contacting the biological sample. In some embodiments, the guide nucleic acid of the Argonaute-guide nucleic acid complex is guided to bind with the RCP by the Argonaute protein. In some embodiments, the Argonaute protein of the Argonaute-guide nucleic acid complex does not cut the RCP after the Argonaute-guide nucleic acid complex contacts the RCP.

### A. Guide Nucleic Acids

In some aspects, the present application provides designs for guide nucleic acids capable of forming DNA-RNA hybrid duplexes or RNA duplexes for binding the Argonaute protein enzyme to at least a portion of a guide target sequence in a target RNA. In some embodiments, the guide nucleic acids may be used to achieve highly sensitive target-primed RCA, resulting in improved sensitivity (number of detected RCPs), signal intensity, increased positional stability in the biological sample, improved accuracy of localization, improved signal to noise, and homogeneity (e.g., narrower size and intensity distributions) compared to RCA reactions using a separate primer.

In some embodiments, the guide nucleic acid comprises RNA. In some embodiments, the guide nucleic acid comprises DNA. In some embodiments, the guide nucleic acid comprises cDNA. In some embodiments, the guide nucleic acid comprises both DNA and RNA. In some embodiments, the guide nucleic acid is single-stranded. In some cases, the guide nucleic acid is a single-stranded DNA (ssDNA) oligonucleotide. In some embodiments, the guide nucleic acid comprises one or more synthetic nucleotides and/or one or more synthetic nucleosides. In some embodiments, the one or more synthetic nucleosides comprise bromodeoxyuridine (BrdU).

In some embodiments, the guide nucleic acid is an RNA molecule, and the Argonaute protein is an RNA-guided Argonaute. In some embodiments, the guide nucleic acid is a DNA molecule, and the Argonaute protein is a DNA-guided Argonaute. In some embodiments, the guide nucleic acid comprises a 5′-phosphate or a 5′-OH. In some embodiments, the guide nucleic acid is at least about 5, at least about 8, at least about 10, at least about 12, at least about 15, at least about 20, or at least about 30 nucleotides in length. In some embodiments, the guide nucleic acid is between about 10 and about 30, about 15 and about 25, about 14 and about 20, about 16 and about 20, about 20 and about 30 nucleotides, or about 25 and about 35 nucleotides in length. In some embodiments, the guide target sequence is at least about 5, at least about 8, at least about 10, at least about 12, at least about 15, at least about 20, or at least about 30 nucleotides in length. In some embodiments, the guide target sequence is between about 10 and about 30, about 15 and about 25, about 14 and about 20, about 16 and about 20, about 20 and about 30 nucleotides, or about 25 and about 35 nucleotides in length. In some embodiments, the guide nucleic acid is 10 to 35 nucleotides in length, 20 to 35 nucleotides in length, 20 to 31 nucleotides in length, 20 to 25 nucleotides in length, 25-35 nucleotides in length, or 26 to 31 nucleotides in length. In some embodiments, the guide nucleic acid is 20 to 30 nucleotides in length. In some embodiments, the guide nucleic acid is 20 to 25 nucleotides in length. In some embodiments, the guide nucleic acid is 26 to 31 nucleotides in length. In some embodiments, the guide nucleic acid is fully complementary to the guide target sequence. In some embodiments, the guide nucleic acid is partially complementary to the guide target sequence. In some embodiments, the guide nucleic acid is at least about 30, about 40, about 50, about 60, about 70, about 80, about 90, about 95, or about 100% complementary to the guide target sequence.

In some embodiments, the 5′ end of the guide nucleic acid comprises a modification. In some embodiments, the guide nucleic acid comprises a modified 5′ residue. In some embodiments, the modified 5′ residue is advantageous for guide nucleic acid recognition of target nucleic acids or for guide nucleic acid binding stability over a guide nucleic acid comprising a non-modified 5′ residue. In some embodiments, the 5′ end of the guide nucleic acid comprises a phosphoryl group. In some embodiments, the 5′ end of the guide nucleic acid comprises a hydroxyl group.

In some embodiments, the guide nucleic acid comprises one or more modified synthetic nucleoside analogues and/or one or more modified synthetic nucleotides. In some embodiments, the 5′ end of the guide nucleic acid comprises a modified synthetic nucleoside analogue. In some embodiments, the 5′ end of the guide nucleic acid comprises a bromodeoxyuridine (BrdU) nucleoside. In some embodiments, the 5′-BrdU nucleoside increases binding stability of the guide nucleic acid compared to the binding stability of a guide nucleic acid that does not comprise a 5′-BrdU (e.g., a guide nucleic acid with the same sequence of bases, but without BrdU.

In some embodiments, the guide nucleic acid comprises a sequence complementary to a variant sequence of a target nucleic acid, such as a variant sequence of interest or an alternative sequence. FIG. 3B shows an example guide nucleic acid 350, comprising a seed region 351 complementary to the gap sequence 342 in a generated RCP, a central region 352, a 3′ supplementary region 353, and an optional 3′ tail 354. The gap sequence 342 is flanked by a first target sequence 341 and a second target sequence 343 in the RCP 340. In some embodiments, the guide nucleic acid 350 comprises a seed region 351 with a nucleotide X′ 391 complementary to the variant sequence of interest X 390. In some embodiments, the seed region 351 is located 5′ to a central region 352 of the guide nucleic acid. In some embodiments, the seed region 351 comprises nucleotide positions 2-8 of the guide nucleic acid 350. In some embodiments, the seed sequence 351 of the guide nucleic acid 350 comprises one or more nucleotides between positions 2 and 8 from the 5′ end of the guide nucleic acid. In some embodiments, the seed sequence 351 comprises a 6-7 nucleotide region. In some embodiments, the seed sequence 351 spans 6-7 nucleotides in the 5′ region of the guide nucleic acid. In some embodiments, the guide nucleic acid 350 also comprises a 3′ supplementary region 353 located 3′ to the central region 352 and the seed region 351. Optionally, in some embodiments, the guide nucleic acid 350 additionally comprises an optional 3′ tail 354. In some embodiments, the guide nucleic acid 350 is between about 14 and 20 nucleotides in length. Optionally, in some embodiments the guide nucleic acid is between about 16 and 20 nucleotides in length. Optionally, in some embodiments, the guide nucleic acid 350 is between 20 and 35 nucleotides in length. In some embodiments, the guide nucleic acid 350 and the Argonaute protein 360 are bound in a complex 380 before contacting the RCP 340. In some embodiments, the guide nucleic acid 350 and the Argonaute protein 360 are bound in a complex 380 before contacting the sample.

### B. Nuclease-Deficient Argonaute Proteins

In some embodiments, the method presented herein further comprises contacting the RCP with an Argonaute-guide nucleic acid complex comprising a guide nucleic acid and an Argonaute protein that does not have cutting activity (i.e., an Argonaute that is slicer-dead). Any suitable Argonaute protein for binding a nucleic acid in a nucleic acid duplex (e.g., within the guide target sequence bound to the guide nucleic acid) without cutting can be used. Generally, Argonaute proteins contain 6 main domains (N-terminal, L1 (Linker 1), PAZ (Piwi-Argonaute-Zwille), L2 (Linker 2), MID (Middle) and PIWI (P-element induced wimpy testis) responsible for binding of a guide nucleic acid and recognition of a guide target sequence. In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule.

In some embodiments, the Argonaute protein is a naturally-occurring protein (e.g., naturally occurs in prokaryotic or eukaryotic cells). In some embodiments, the Argonaute protein is not a naturally-occurring protein (e.g., a variant or mutant protein). In some embodiments, the Argonaute protein is a recombinant protein. In some embodiments, the Argonaute protein is genetically engineered (such as an Argonaute protein described in WO 2019/222036, which is hereby incorporated by reference in its entirety). In some embodiments, the Argonaute protein is a slicer-dead Argonaute protein, meaning that it lacks cutting activity or is nuclease-dead. In some embodiments, the Argonaute protein has been modified (e.g., genetically engineered or mutated) to lack cutting activity. In some embodiments, lacking cutting activity means that the Argonaute protein is not capable of cutting a target nucleic acid. In some embodiments, lacking cutting activity means that the Argonaute protein does not cut the target nucleic acid. In some embodiments, an Argonaute protein that naturally lacks cutting activity or that has been modified to lack cutting activity is a slicer-dead Argonaute.

In some embodiments, the Argonaute protein is a eukaryotic Argonaute protein. Generally, eukaryotic Argonaute proteins can mediate binding of a target RNA with a guide nucleic acid of RNA. In some embodiments, an Argonaute protein is of plant, algal, fungal (e.g., yeast), or animal (e.g., human, rodent, fruit fly, cnidarian, echinoderm, nematode, fish, amphibian, reptile, bird, etc.) origin. In some embodiments, the Argonaute protein is a eukaryotic Argonaute protein that has been modified to lack cutting activity.

In some embodiments, the Argonaute protein is a slicer-dead Ago1, Ago2, Ago3, Ago4, PIWI 1, PIWIL 2, PIWI 3, or PIWI 4 (such as the Argonaute proteins described in WO 2007/048629, which is hereby incorporated by reference in its entirety). In some embodiments, the Argonaute protein is Ago2. In some embodiments, the Ago2 is Drosophila Ago2. In some embodiments, the Argonaute protein is a recombinant Drosophila Argonaute protein. In some embodiments, the Argonaute protein is expressed in a mammalian cell line. In some embodiments, the Argonaute protein is a Drosophila Argonaute protein expressed in a mammalian cell line. In some embodiments, a Drosophila Argonaute protein is expressed using a method such that a loading complex specific to Drosophila species is not provided to obtain guide-free proteins. In some embodiments, the Argonaute protein is a purified recombinant Drosophila Argonaute protein. In some embodiments, the Argonaute protein is expressed in an insect cell line, such as a Schneider 2 (S2) cell line. In some embodiments, the Argonaute protein is a Drosophila Argonaute protein expressed in an insect cell line, such as a S2 cell line. In some embodiments, the Drosophila Argonaute protein is loaded with the guide nucleic acid prior to contacting the biological sample.

In some embodiments, the slicer-dead Argonaute protein is a eukaryotic Argonaute protein from a mammalian organism. In some embodiments, the mammalian Argonaute protein is selected from mammalian AGO1, AGO2, AGO3, and AGO4. In some embodiments, the mammalian Argonaute protein is a human Argonaute protein. In some embodiments, the human Argonaute protein is a human AGO1 or AGO4 protein which naturally lacks slicer activity (See Faehnle et al. The making of a slicer: activation of a human Argonaute-1. Cell Reports 2015 Jun. 27, 3(6): 1901-1909, which is hereby incorporated by reference in its entirety). In some embodiments, the human Argonaute protein is a human AGO2 protein that has been modified to lack slicer activity (See McGeary et al., The Biochemical Basis of microRNA Targeting Efficacy. Science 2019 Dec. 20; 366(6472): eaav1741., which is hereby incorporated by reference in its entirety). In some embodiments, the human Argonaute protein is a human AGO3 protein that has been modified to lack slicer activity.

In some embodiments, the Argonaute protein is used as described herein for binding a target sequence (e.g., complementary to the variant sequence in the RCP) at a temperature at which slicer activity of the Argonaute protein is not active. In some examples, an Argonaute protein derived from Thermus thermophilus (dTtAgo) is used to bind a target sequence at about 30° C. (See Shin et al, “Quantification of purified endogenous miRNAs with high sensitivity and specificity.” Nature Commun 11:6033 (2020), which is herein incorporated by reference in its entirety).

In some embodiments, the slicer-dead Argonaute protein is a prokaryotic Argonaute protein or a variant thereof. Generally, prokaryotic Argonaute proteins can mediate binding of a target RNA with a guide oligonucleotide. In some cases, the prokaryotic Argonaute protein uses RNA as a guide oligonucleotide. In some cases, the prokaryotic Argonaute protein uses DNA as a guide oligonucleotide. In some embodiments, the slicer-dead Argonaute protein is a prokaryotic Argonaute protein that has been modified to lack cutting activity.

In some embodiments, the slicer-dead Argonaute protein is a modified Nitratireductor (optionally Nitratirereductor sp. XY-223), Enhydrobacter (optionally Enhydrobacter aerosaccus), Mesorhizobium (optionally Mesorhizobium sp. CNPSo 3140), Hyphomonas (optionally Hyphomonas sp. T16B2), Pseudooceanicola (optionally Pseudooceanicola lipolyticus), Tateyamaria (optionally Tateyamaria omphalii), Bradyrhizobium (optionally Bradyrhizobium sp. ORS 3257), Dehalococcoides (optionally Dehalococcoides mccartyi), Chroococcidiopsis (optionally Chroococcidiopsis cubana), Runella (optionally Runella slithyformis), Roseivirga (optionally Rosevirga seohaensis), Spirosoma (optionally Spirosoma endophyticum), Pedobacter (optionally Pedobacter yonginense, Pedobacter insulae, or Pedobacter nyackensis), Planctomycetes bacterium (optionally Planctomycetes bacterium TBKIr or Planctomycetes bacterium V6), Dyadobacter (optionally Dyadobacter sp. QTA69), Mucilaginibacter (optionally Mucilaginibacter gotjawali, Mucilaginibacter polytichastri or Mucilaginibacter paludis), Hydrobacter (optionally Hydrobacter penzbergensis), Chitinophaga (optionally Chitinophaga costaii), Cytophagaceae bacterium (optionally Cytophagaceae bacterium SJW1-29), Emticicia (optionally Emticicia oligotrophica), Runella (optionally Runella sp. YX9), or Spirosoma (optionally Spirosoma pollinicola) Argonaute protein (See Li et al., “A programmable pAgo nuclease with RNA target preference from the psychrotolerant bacterium Mucilaginibacter paludis” Nucleic Acids Res. 2022 May 20; 50(9): 5226-5238; Lisitskaya et al., “Programmable RNA targeting by bacterial Argonaute nucleases with unconventional guide binding and cleavage specificity.” Nat Commun. 2022 Aug. 8; 13(1): 4624; Sun et al., “An Argonaute from Thermus parvatiensis exhibits endonuclease activity mediated by 5′ chemically modified DNA guides.” Acta Biochim Biophys Sin (Shanghai). 2022 May 25; 54(5): 686-695; U.S. Pat. No. 10,253,311; U.S. Ser. No. 15/089,243; U.S. Ser. No. 17/575,957; U.S. Ser. No. 17/854,897; and WO 2022/222920 each of which herein incorporated by reference in their entireties) that has been modified to lack cutting activity. In some embodiments, the slicer-dead Argonaute protein is from Thermus thermophilus. In some embodiments, the slicer-dead Argonaute protein is from Marinitoga piezophile (See Lapinaite et al, “Programmable RNA recognition by a CRISPR-associated Argonaute.” PNAS 2018 Mar. 27; 115(13): 3368-3373, which is herein incorporated by reference in its entirety). In some embodiments, the slicer-dead Argonaute protein is from Rhobacter sphaeroidis (See Miyoshi et al, “Structural basis for the recognition of guide RNA and target DNA heteroduplex by Argonaute.” Nature Comm 2016; 7:11846, which is herein incorporated by reference in its entirety). In some embodiments, the slicer-dead Argonaute protein is from Thermomyces thermophilus (such as an Argonaute protein described in patent application no. CN202210082114, the content of which is herein incorporated by reference in its entirety). In some embodiments, the slicer-dead Argonaute protein is from Vanderwaltozyma polyspora (also known as Kluyveromyces polysporus) (such as an Argonaute protein described in WO 2018/112336, the content of which is herein incorporated by reference in its entirety). In some embodiments, the slicer-dead Argonaute protein is a modified Argonaute protein from Clostridium perfringens (CpAgo) or an Argonaute protein from Intestinibacter bartlettii (IbAgo) that lacks cutting activity (See Cao et al, Argonaute proteins from human gastrointestinal bacteria catalyze DNA-guided cleavage of single- and double-stranded DNA at 37 C. Cell Discovery 2019 5(38), which is hereby incorporated by reference in its entirety). In some embodiments, the slicer-dead Argonaute protein is a modified Argonaute protein from Clostridium butyricum (CbAgo) that lacks cutting activity (See Hegge et al. DNA-guided DNA cleavage at moderate temperatures by Clostridium butyricum Argonaute. BioRXIV 2019, https://doi.org//10.1101/534206 and Kuzmenko et al. Programmable DNA cleavage by Ago nucleases from mesophilic bacteria Clostridium butyricum and Limnothrix rosea. BioRXIV 2019, https://doi.org//10.1101/558684, both of which are hereby incorporated by reference in their entirety).

In some embodiments, the slicer-dead Argonaute protein is a variant of a DNA-binding Argonaute protein. In some embodiments, the Argonaute is a DNA-guided Pyrococcus furiosus (PfAgo) that binds single- and/or double-stranded DNA (See Swarts et al, “Argonaute of the archaeon Pyrocuccus furiosus is a DNA-guided nuclease that targets cognate DNA.” Nucleic Acids Research Volume 43, Issue 10, 26 May 2015, Pages 5120-5129, which is herein incorporated by reference in its entirety). In some embodiments, the Argonaute protein has been modified to lack cutting activity of an RNA substrate via selection and/or directed evolution.

In some embodiments, the slicer-dead Argonaute protein comprises one or more amino acid substitutions compared to any of the species of Argonaute protein described herein. In certain embodiments, the one or more amino acid substitutions are conservative substitutions. In some aspects, conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine(S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g., U.S. Pat. No. 8,562,989; pages 13-15 “Biochemistry” 2^{nd} ED. Lubert Stryer ed (Stanford University); Henikoff et al., PNAS 1992 Vol 89 10915-10919; Lei et al., J Biol Chem 1995 May 19; 270(20): 11882-6, each of which is herein incorporated by reference in its entirety).

An amino acid substitution may include replacement of one amino acid in a polypeptide with another amino acid. Amino acid substitutions may be introduced to generate a modified Argonaute protein as described herein.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some contexts, conservative substitutions can involve the exchange of a member of one of these classes for another member of the same class. In some contexts, non-conservative amino acid substitutions can involve exchanging a member of one of these classes for another class. In some contexts, particular substitutions can be considered “conservative” or “non-conservative” depending on the stringency and context and environment of the particular residue in primary, secondary and/or tertiary structure of the protein.

In some embodiments, the Argonaute is nuclease-deficient (i.e., lacks slicer activity). In some embodiments, the Argonaute is not capable of cutting the RCP. In some embodiments, the Argonaute protein does not cut the RCP. In some embodiments, the Argonaute protein is an Argonaute protein that lacks slicer activity. In some embodiments, the Argonaute protein is an Argonaute protein that has been modified (i.e., selectively mutated) to lack slicer activity. In some embodiments, the modified, nuclease-deficient Argonaute protein comprises one or more inactivating mutations in a PIWI and/or PAZ domain of the Argonaute protein.

In some embodiments, the slicer-dead Argonaute protein is an RNA-guided Argonaute, and the guide nucleic acid is an RNA molecule. In some embodiments, the slicer-dead Argonaute protein is a eukaryotic Argonaute protein. In some embodiments, the slicer-dead Argonaute protein is a DNA-guided Argonaute, and the guide nucleic acid is a DNA molecule. In some embodiments, the slicer-dead Argonaute protein is a prokaryotic Argonaute protein. In some embodiments, the guide nucleic acid comprises a 5′-phosphate or a 5′-OH. In some embodiments, the nuclease-deficient Argonaute protein is Ago1, Ago3, or Ago4. In some embodiments, the nuclease-deficient Argonaute protein is a Drosophila Argonaute protein or a derivative or variant thereof. In some embodiments, the nuclease-deficient Argonaute protein is a nuclease-deficient Argonaute derived from Thermus thermophilus (dTtAgo).

In some embodiments, the nuclease-deficient Argonaute is a nuclease-deficient Argonaute derived from Marinitoga piezophile (MpAgo). In some embodiments, the nuclease-deficient Argonaute is a MpAgo that has been additionally modified to lack slicer activity. In some embodiments, the MpAgo protein forms a complex with a 5′-hydroxylated guide nucleic acid to form a MpAgo-guide nucleic acid complex. In some embodiments, the guide nucleic acid of the MpAgo-guide nucleic acid complex comprises a 5′-BrdU (e.g., as described in Lapinaite et al., “Programmable RNA recognition using a CRISPR-associated Argonaute”, PNAS 2018) for increased binding stability of MpAgo-guide nucleic acid complex. In some embodiments, a seed region of the MpAgo-guide nucleic acid complex comprises a noncanonical seed region comprising nucleotides 5-15 of the guide nucleic acid. In some embodiments, the noncanonical 5-15 nucleotide seed region of the MpAgo-guide nucleic has full complementarity to the target nucleic acid sequence. In some embodiments, the MpAgo-guide nucleic acid complex is used to detect a SNV at the position of the target RNA that is complementary to nucleic acids 6 or 7 of the guide nucleic acid. In some embodiments, the MpAgo-guide nucleic acid can detect modified nucleotides in the target RNA at positions complementary to nucleotides 6-7 of the guide nucleic acid.

In some embodiments, the slicer-dead Argonaute protein is conjugated to a nucleic acid barcode sequence. In some embodiments, the nucleic acid barcode sequence is detected in situ with a detectably labeled probe and/or an intermediate probe comprising a barcode sequence complementary to the barcode sequence or a portion thereof. In some embodiments, the method comprises detecting the nucleic acid barcode sequence by sequencing all or a portion of the nucleic acid barcode sequence (e.g., sequencing-by-synthesis, sequencing-by-binding, sequencing-by-avidity, or sequencing-by-ligation). In some embodiments, the slicer-dead Argonaute protein comprises a detectable moiety (e.g., a fluorescent label). Examples of detectable moieties (also referred to as detectable labels) are described in Section VII. In some embodiments, the method comprises detecting the detectable moiety of the slicer-dead Argonaute protein. In some embodiments, the slicer-dead Argonaute protein is contacted with the biological sample in a pre-formed complex with the guide nucleic acid, and the nucleic acid barcode sequence or detectable label attached to the slicer-dead Argonaute protein corresponds to a sequence of the guide nucleic acid. In some embodiments, the detectable label or barcode sequence is selected such that it identifies a sequence of the guide nucleic acid that determines binding of the guide nucleic acid/Argonaute complex to a complementary sequence. In some embodiments, the slicer-dead Argonaute protein is not conjugated to a nucleic acid barcode sequence that identifies a guide nucleic acid. In some embodiments, the slicer-dead Argonaute protein is not detectably labeled. In some embodiments, the slicer-dead Argonaute protein is capable of forming a complex with different guide nucleic acids.

### C. In Situ Detection of Argonaute-Guide Nucleic Acid Complexes

In some embodiments, the method disclosed herein further comprises contacting an RCP generated in a biological sample with an Argonaute-guide nucleic acid complex comprising a nuclease-deficient Argonaute protein (i.e., a slicer-dead Argonaute protein that lacks cutting activity) and a guide nucleic acid as described in the preceding sections, for example, as shown in FIG. 2. In some embodiments, the contacting comprises contacting the biological sample with a plurality of nuclease-deficient Argonaute-guide nucleic acid complexes wherein the Argonaute-guide nucleic acid complexes comprise distinct guide nucleic acids with distinct seed sequences corresponding to a plurality of variant sequences, and each different Argonaute-guide nucleic acid complex comprises a different detectable label. In some embodiments, the Argonaute-guide nucleic acid complex binds to the RCP at a variant sequence, enabling detection of the variant sequence in the biological sample. In some instances, imaging is performed to detect the complex formed between the Argonaute protein, the guide nucleic acid, and the RCP in the biological sample.

An example of a method of detecting bound Argonaute-guide nucleic acid complexes bound to an RCP at a location in the biological sample is shown in FIG. 2 (righthand panel). In some embodiments, a first Argonaute-guide nucleic acid complex 251 for detecting a variant sequence of interest X 270 comprises a first nuclease-deficient Argonaute protein 250, a first guide nucleic acid 260 comprising a complementary sequence X′ 271, and a first detectable fluorescent label 280. In some embodiments, the first Argonaute-guide nucleic acid complex 251 binds to a first RCP 290 comprising multiple copies of the variant sequence of interest X 270. In some embodiments, a second Argonaute-guide nucleic acid complex 252 for detecting an alternative variant sequence Y 272 comprises a second nuclease-deficient Argonaute protein 253, a second guide nucleic acid 261 comprising a complementary sequence Y′ 273, and a second detectable fluorescent label 281. In some embodiments, the second Argonaute-guide nucleic acid complex 252 binds to a second RCP 291 comprising multiple copies of the alternative variant sequence Y 272 at the alternative variant sequence. In some embodiments, the biological sample comprising RCPs 290, 291 is imaged to detect the first fluorophore 280 of the first bound Argonaute-guide nucleic acid complex 251 at a location in the biological sample and/or the second fluorophore 281 of the second bound Argonaute-guide nucleic acid complex 252 at a location in the biological sample. In some embodiments, detection of the first fluorophore 280 indicates the presence of the variant sequence of interest X 270 at a location in the biological sample. In some embodiments, detection of the second fluorophore 281 indicates the presence of the alternative variant sequence Y 272 at a location in the biological sample.

In some embodiments, a nuclease-deficient Argonaute protein of an Argonaute-guide nucleic acid complex comprises a detectable moiety, for example, as shown in FIG. 2 280, 281. In some embodiments, the guide nucleic acid of the Argonaute-guide nucleic acid complex comprises a detectable moiety. In some embodiments, the detectable moiety comprises a detectable fluorescent label. In some embodiments, a plurality of detectable fluorescent labels are used to label a plurality of Argonaute-guide nucleic acid complexes with guide nucleic acids corresponding to a plurality of variant sequences such that, for example, the presence of a variant sequence of interest in the biological sample is indicated by the presence of a first fluorescent label (e.g., a green fluorophore), and the presence of an alternative sequence in the biological sample is indicated by the presence of a second fluorescent label (e.g., a red fluorophore), and the first fluorescent label and second fluorescent label can be easily and reliably visually discriminated from one another in a decoding step.

In some embodiments, the guide nucleic acid of the Argonaute-guide nucleic acid complex comprises a sequence complementary to a variant sequence in the RCP, allowing for detection of the variant sequence in the RCP when the Argonaute-guide nucleic acid complex is bound to the RCP at the variant sequence, for example, as shown in FIG. 3B. In some embodiments, the guide nucleic acid 350 comprises a seed region 351 comprising a seed sequence 391 complementary to the variant sequence 390. In some embodiments, the seed sequence 351 of the guide nucleic acid 350 comprises one or more nucleotides between positions 2 and 8 from the 5′ end of the guide nucleic acid. In some embodiments, the seed sequence 351 comprises 6-7 consecutive nucleotides of the 5′ region of the guide nucleic acid. In some embodiments, the seed sequence 351 spans 6-7 nucleotides within the 5′ region of the guide nucleic acid.

In some embodiments, the guide nucleic acid 350 of the Argonaute-guide nucleic acid complex comprises an optional 3′ tail sequence 354, wherein the method comprises contacting the RCP 340 in the biological sample with a detectably labeled probe that binds directly or indirectly to the 3′ tail sequence 354 of the guide nucleic acid, and wherein detecting the Argonaute-guide nucleic acid complex 380 bound to the RCP in the biological sample comprises detecting the detectably labeled probe bound directly or indirectly to the 3′ tail sequence 354 of the guide nucleic acid. In some embodiments, the detectably labeled probe is a detectably labeled probe of a plurality of detectably labeled probes, wherein the method comprises contacting sequential cycles of binding detectably labeled probes directly or indirectly with the 3′ tail sequence of the guide nucleic acid or sub-sequences thereof.

In some embodiments, an Argonaute-guide nucleic acid complex is labeled with a detectable moiety such that it can be directly detected in situ in a biological sample. In some embodiments, the Argonaute protein of the Argonaute-guide nucleic acid complex is labeled with the detectable moiety. In some embodiments, the guide nucleic acid of the Argonaute-guide nucleic acid complex is labeled with the detectable moiety. In some embodiments, the detectable moiety is a fluorescent dye. In some embodiments, the fluorescently labeled guide nucleic acid 350 of the Argonaute-guide nucleic acid complex 380 binds to a variant sequence 390 in an RCP generated in the biological sample and the fluorescent moiety can then be detected at a location in the biological sample.

In some aspects, the provided methods involve analyzing, e.g., detecting or determining, one or more nucleic acid sequences such as gap sequences in target nucleic acids. In some embodiments, the detection or determination comprises binding one or more Argonaute-guide nucleic acid complexes to nucleic acid molecules such as RCPs (e.g., described in Section IV). In some cases, the analysis is performed on one or more images captured, and may comprise processing the image(s) and/or quantifying signals observed. In some embodiments, the analysis comprises detecting a sequence (e.g., a gap sequence) present in the sample. In some embodiments, the analysis comprises quantification of puncta (e.g., if amplification products are detected). In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further comprises displaying the information from the analysis or detection step. In some embodiments, software may be used to automate the processing, analysis, and/or display of data.

In some embodiments, a detectably labeled probe can hybridize to a detectable region in an Argonaute-guide nucleic acid complex disclosed herein. In some embodiments, the detectable region is in the guide nucleic acid. In some embodiments, the detectable region is in an optional 3′ tail of the guide nucleic acid. In some embodiments, the detectable region is in the Argonaute protein.

In some embodiments, provided herein are methods involving the use of one or more probes for analyzing one or more target nucleic acid(s), such as variant sequences in one or more target nucleic acids present in a cell or a biological sample, such as a tissue sample. In some embodiments, the probes can include a plurality of detectably labeled probes for combinatorially decoding the barcode regions in the Argonaute-guide nucleic acid complexes bound to variant sequences in the RCPs, and/or the barcode regions in the RCPs. In some embodiments, the probes can include a plurality of detectably labeled probes for combinatorially decoding the barcode regions in the Argonaute-guide nucleic acid complexes and/or the RCPs. Using sequential probe hybridization, the provided embodiments can be employed for in situ detection of variant sequences in target nucleic acids in a cell, e.g., in cells of a biological sample or a sample derived from a biological sample, such as a tissue section on a solid support, such as on a transparent slide.

In some aspects, provided herein are in situ assays using microscopy as a readout, e.g., hybridization, or other detection or determination methods involving an optical readout. In some aspects, detection or determination of a sequence of one, two, three, four, five, or more nucleotides of a gap sequence in a target nucleic acid is performed in situ in a cell and/or in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product or hybridization complex). In some embodiments, the detecting comprises contacting the RCPs with a detectably labelled Argonaute-guide nucleic acid complex comprising a guide nucleic acid comprising a complement of the amplification product or portion thereof, and then detecting the detectably labelled Argonaute-guide nucleic acid complex at the location in the biological sample. In some embodiments, a provided method is quantitative and preserves the spatial information within a tissue sample without physically isolating cells or using homogenates. In some embodiments, the present disclosure provides methods for high-throughput profiling of target nucleic acids in situ in a large number of cells, tissues, organs or organisms.

In some embodiments, the provided methods comprise imaging the amplification product (e.g., RCA product) of a probe or probe set (e.g., as described in Section IV) and the bound Argonaute-guide nucleic acid complex (e.g., as described in Section V) via binding of detectably labeled probes (e.g., detection oligonucleotides each comprising a fluorescent label), and detecting the detectable labels, for instance, in sequential probe hybridization and detection cycles.

A method disclosed herein comprises contacting a biological sample with an Argonaute-guide nucleic acid complex that binds to an RCP at a variant sequence, wherein the RCP is generated in the biological sample. In some embodiments, the guide nucleic acid of the Argonaute-guide nucleic acid complex comprises a barcode region and a seed region for interrogating the variant sequence in the RCP. In some embodiments, the barcode region in the Argonaute-guide nucleic acid complex is associated with the variant sequence in the target nucleic acid. In some embodiments, the barcode region in the Argonaute-guide nucleic acid complex is not associated with the variant sequence in the target RNA. In some embodiments, the method further comprises removing molecules of Argonaute-guide nucleic acid complexes that are not bound due to a mismatch between the guide nucleic acid and the variant sequence in the RCP (for example, performing one or more wash steps). In some embodiments, the method further comprises contacting the biological sample with detectably labeled probes in sequential cycles. In some embodiments, in each cycle, a detectably labeled probe is directly or indirectly bound to a barcode sequence in the barcode region and an observed signal (e.g., an observed level of signal) associated with the detectably labeled probe is recorded at a location in the biological sample, thereby generating a signal code sequence corresponding to the barcode region. In some embodiments, the signal code sequence comprises the observed signal (e.g., an observed level of signal) recorded at the location in each of the sequential cycles. In some embodiments, the method further comprises using the signal code sequence to identify the variant sequence of the target nucleic acid at the location in the biological sample.

In some embodiments, the biological sample is contacted with a plurality of Argonaute-guide nucleic acid complexes each comprising a different guide nucleic acid seed region complementary to different variant sequence of the target RNA and a different barcode region corresponding to the different variant sequence. In some embodiments, each set of guide nucleic acids comprises a different seed region for a different variant sequence of the target nucleic acid and a different barcode region corresponding to the different variant sequence. In some embodiments, the barcode region is in the 3′ tail region of the guide nucleic acid. In some embodiments, in each cycle, the detectably labeled probe is hybridized to an intermediate probe which in turn hybridizes to the barcode region. In some embodiments, in two or more of the sequential cycles, the intermediate probes are hybridized to the same barcode sequence or different barcode sequences in the barcode region. In some embodiments, in each of the sequential cycles, the intermediate probe is hybridized to the same barcode sequence or a different barcode sequence in the barcode region. In some embodiments, in each cycle, the detectably labeled probe is hybridized to the barcode region. In some embodiments, in two or more of the sequential cycles, the detectably labeled probes are hybridized to different barcode sequences in the barcode region. In some embodiments, in each of the sequential cycles, the detectably labeled probe is hybridized to a different barcode sequence in the barcode region. In some embodiments, the barcode region comprises two or more non-overlapping barcode sequences. In some embodiments, the barcode region comprises two or more overlapping barcode sequences. In some embodiments, each pair of adjacent barcode sequences in the barcode region are partially overlapping.

As shown in FIG. 2, lefthand panel, in some embodiments the method disclosed herein comprises contacting the biological sample with a probe or probe set. In some embodiments, the probe or probe set comprises a first probe region and a second probe region that bind to a first target sequence and a second target sequence, respectively, in a target nucleic acid (e.g., an RNA) in the biological sample, and a complement of a first barcode region. In some embodiments, the first barcode region comprises one or more barcode sequences associated with the target RNA or a sequence thereof but not associated with the variant sequence. In some embodiments, the first barcode region comprises two or more non-overlapping barcode sequences. In some embodiments, the first barcode region comprises two or more overlapping barcode sequences. In some embodiments, each pair of adjacent barcode sequences in the first barcode region are partially overlapping. In some embodiments, the first and second target sequences flank a gap sequence in the target RNA, and wherein the gap sequence comprises a variant sequence. In some embodiments, the method further comprises performing a gap-fill reaction on the probe or probe set to generate a gap-filled probe or probe set. In some embodiments, the method further comprises circularizing the gap-filled probe or probe set to generate a circularized probe comprising a gap-filled region complementary to the gap sequence. In some embodiments, the method further comprises using a polymerase to amplify the circularized probe to generate a rolling circle amplification product (RCP) in the biological sample.

In some embodiments, the method further comprises contacting the biological sample with an Argonaute-guide nucleic acid complex that binds to the RCP at a variant sequence. In some embodiments, the guide nucleic acid of the Argonaute-guide nucleic acid complex comprises a seed region for binding to a variant sequence (i.e., either a seed region for binding to a variant sequence of interest, or a seed region for binding to an alternative variant sequence) in the RCP, and a second barcode region. In some embodiments, the second barcode region consists of one barcode sequence corresponding to a base selected from the group consisting of A, T, C, and G. In some embodiments, the barcode sequence corresponding to the base is common among a plurality of second probes or probe sets targeting different target RNAs or different sequences of the same target RNA. In some embodiments, the second barcode region is associated with the variant sequence in the target RNA.

In some embodiments, the method further comprises removing molecules of Argonaute-guide nucleic acid complexes that are not bound due to a mismatch with the variant sequence in the RCP. In some embodiments, the method further comprises contacting the biological sample with detectably labeled probes in sequential cycles. In some embodiments, in each cycle, a detectably labeled probe is directly or indirectly bound to a barcode sequence in the first or second barcode region and a signal or absence thereof associated with the detectably labeled probe is recorded at a location in the biological sample, thereby generating a signal code sequence corresponding to the first and second barcode regions. In some embodiments, in each cycle, the detectably labeled probe is hybridized to the first barcode region or the second barcode region. In some embodiments, in two or more of the sequential cycles, the detectably labeled probes are hybridized to different barcode sequences in the first barcode region or different barcode sequences the second barcode region. In some embodiments, in each of the sequential cycles for decoding the first barcode region, the detectably labeled probe is hybridized to a different barcode sequence in the first barcode region. In some embodiments, each pair of adjacent barcode sequences in the first barcode region are partially overlapping. In some embodiments, in each cycle, the detectably labeled probe is hybridized to an intermediate probe which in turn hybridizes to the first barcode region or the second barcode region. In some embodiments, in two or more of the sequential cycles, the intermediate probes are hybridized to the same barcode sequence or different barcode sequences in the first barcode region or the second barcode region. In some embodiments, in each of the sequential cycles for decoding the first barcode region, the intermediate probe is hybridized to the same barcode sequence or a different barcode sequence in the first barcode region.

In some embodiments, the signal code sequence comprises the observed signal (e.g., level of signal) recorded at the location in each of the sequential cycles. In some embodiments, the method provided herein further comprises using the signal code sequence to identify the variant sequence of the target RNA at the location in the biological sample.

In some embodiments, a signal associated with a detectably labeled oligonucleotide is measured and quantitated. The terms “label” and “detectable label” comprise a directly or indirectly detectable moiety that is associated with (e.g., conjugated to) a molecule to be detected, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), each of which is herein incorporated by reference in its entirety. In some embodiments, techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), each of which is herein incorporated by reference in its entirety. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, each of which is herein incorporated by reference in its entirety. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, a guide nucleic acid disclosed herein comprises one or more detectably labeled, e.g., fluorescent, nucleotides. In some embodiments, the one or more detectably labelled nucleotides are incorporated in the guide nucleic acid. Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For examples of methods for custom synthesis of nucleotides having other fluorophores, see, Henegariu et al. (2000) Nature Biotechnol. 18:345, which is herein incorporated by reference in its entirety.

Other fluorophores available for post-synthetic attachment (e.g., to an Argonaute protein) comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62, which is herein incorporated by reference in its entirety).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160, each of which is hereby incorporated by reference in its entirety. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of an RCP disclosed herein. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of an RCP disclosed herein. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

### III. Argonaute-Mediated Obliteration of RCA Products Using Nuclease-Active Argonaute-Guide Nucleic Acid Complexes

In some embodiments, herein is provided a method for detecting a variant sequence of interest in an RCP generated from a gap-filled circularized probe in a biological sample, further comprising cutting the RCP at a variant sequence (e.g., either at a variant sequence of interest or at an alternative variant sequence) with a slicer-active Argonaute-guide nucleic acid complex comprising a slicer-active Argonaute protein (i.e., an Argonaute protein that is capable of cutting a target nucleic acid) and a guide nucleic acid comprising a seed sequence that is complementary to the variant sequence of interest or the alternative variant sequence.

In some embodiments, Argonaute-guide nucleic acid complexes comprising nuclease-active Argonaute proteins are capable of cutting at a precise location in a variant sequence of a target nucleic acid (e.g., a rolling circle amplification product generated from a gap-fill circularizable probe or probe set comprising a complement of the variant sequence in the target nucleic acid, such that the rolling circle amplification product comprises multiple copies of the variant sequence). In some embodiments, Argonaute-mediated obliteration of an abundant variant sequence may reduce optical crowding from fluorescent moieties at a location in the biological sample during imaging and detection. In some embodiments, Argonaute-mediated obliteration of RCPs with slicer-active Argonaute-guide nucleic acid complexes may be useful for reducing signals from a highly abundant alternative sequence (e.g., a wild-type sequence) to enable or improve detection of a less abundant variant sequence of interest (e.g., a mutant sequence).

### A. Guide Nucleic Acids

In some embodiments, herein is disclosed a method for discriminating variant sequences in RCPs generated in a biological sample using slicer-active Argonaute-guide nucleic acid complexes, wherein the guide nucleic acids are capable of forming complexes with complementary target nucleic acids, enabling cutting by the slicer-active Argonaute protein. In some embodiments, the guide nucleic acids of the present invention are capable of forming DNA-RNA, DNA, or RNA duplexes for cutting by the Argonaute protein enzyme in at least a portion of a guide target sequence in a target nucleic acid.

In some embodiments, the guide nucleic acid comprises RNA. In some embodiments, the guide nucleic acid comprises DNA. In some embodiments, the guide nucleic acid comprises cDNA. In some embodiments, the guide nucleic acid comprises both DNA and RNA. In some embodiments, the guide nucleic acid is single-stranded. In some cases, the guide nucleic acid is a single-stranded DNA (ssDNA) oligonucleotide. In some embodiments, the guide nucleic acid comprises one or more synthetic nucleotides and/or one or more synthetic nucleosides. In some embodiments, the one or more synthetic nucleosides comprise bromodeoxyuridine (BrdU).

In some embodiments, the guide nucleic acid is an RNA molecule, and the Argonaute protein is an RNA-guided Argonaute. In some embodiments, the guide nucleic acid is a DNA molecule, and the Argonaute protein is a DNA-guided Argonaute. In some embodiments, the guide nucleic acid comprises a 5′-phosphate or a 5′-OH. The guide nucleic acid is at least about 5, at least about 8, at least about 10, at least about 12, at least about 15, at least about 20, or at least about 30 nucleotides in length. In some embodiments, the guide nucleic acid is between about 10 and about 30, about 15 and about 25, about 14 and about 20, about 16 and about 20, about 20 and about 30 nucleotides, or about 25 and about 35 nucleotides in length. In some embodiments, the guide target sequence is at least about 5, at least about 8, at least about 10, at least about 12, at least about 15, at least about 20, or at least about 30 nucleotides in length. In some embodiments, the guide target sequence is between about 10 and about 30, about 15 and about 25, about 14 and about 20, about 16 and about 20, about 20 and about 30 nucleotides, or about 25 and about 35 nucleotides in length. In some embodiments, the guide nucleic acid is fully complementary to the guide target sequence. In some embodiments, the guide nucleic acid is partially complementary to the guide target sequence. In some embodiments, the guide nucleic acid is at least about 30, about 40, about 50, about 60, about 70, about 80, about 90, about 95, or about 100% complementary to the guide target sequence.

In some embodiments, the guide nucleic acid is 10 to 35 nucleotides in length, 20 to 35 nucleotides in length, 20 to 31 nucleotides in length, 20 to 25 nucleotides in length, 25-35 nucleotides in length, or 26 to 31 nucleotides in length. In some embodiments, the guide nucleic acid is 20 to 30 nucleotides in length. In some embodiments, the guide nucleic acid is 20 to 25 nucleotides in length. In some embodiments, the guide nucleic acid is 26 to 31 nucleotides in length.

In some embodiments, the 5′ end of the guide nucleic acid comprises a modification. In some embodiments, the guide nucleic acid comprises a modified 5′ residue. In some embodiments, the modified 5′ residue is advantageous for guide nucleic acid recognition of target nucleic acids or for guide nucleic acid binding stability over a guide nucleic acid comprising a non-modified 5′ residue. In some embodiments, the 5′ end of the guide nucleic acid comprises a phosphoryl group. In some embodiments, the 5′ end of the guide nucleic acid comprises a hydroxyl group.

In some embodiments, the guide nucleic acid comprises one or more modified synthetic nucleoside analogues and/or one or more modified synthetic nucleotides. In some embodiments, the 5′ end of the guide nucleic acid comprises a modified synthetic nucleoside analogue. In some embodiments, the 5′ end of the guide nucleic acid comprises a bromodeoxyuridine (BrdU) nucleoside. In some embodiments, the 5′-BrdU nucleoside increases binding stability of the guide nucleic acid compared to the binding stability of a guide nucleic acid with similar seed sequence complementarity to the target nucleic acid that does not comprise a 5′-BrdU.

In some embodiments, the guide nucleic acid comprises a sequence complementary to the alternative variant sequence. In some embodiments, the guide nucleic acid may additionally comprise a sequence complementary to part or all of one or both of the target sequences flanking the variant sequence. FIG. 3B shows an example of a guide nucleic acid 350, comprising a seed region 351 complementary to the gap sequence 342, a central region 352, a 3′ supplementary region 353, and an optional 3′ tail 354. In some embodiments, the guide nucleic acid 350 comprises a seed region 351 with a nucleotide X′ 391 complementary to the variant sequence of interest X 390. In some embodiments, the seed region 351 is located 5′ to a central region 352 of the guide nucleic acid. In some embodiments, the seed region 351 is located in nucleotide positions 2-8 of the guide nucleic acid 350. In some embodiments, the seed sequence 351 of the guide nucleic acid 350 comprises one or more nucleotides between positions 2 and 8 from the 5′ end of the guide nucleic acid. In some embodiments, the seed sequence 351 comprises a 6-7 nucleotide region. In some embodiments, the seed sequence 351 spans 6-7 nucleotides in the 5′ region of the guide nucleic acid. In some embodiments, the guide nucleic acid 350 also comprises a 3′ supplementary region 353 located 3′ to the central region 352 and the seed region 351. Optionally, in some embodiments, the guide nucleic acid 350 additionally comprises an optional 3′ tail 354. In some embodiments, the guide nucleic acid 350 is between about 14 and 20 nucleotides in length. Optionally, in some embodiments the guide nucleic acid is between about 16 and 20 nucleotides in length. Optionally, in some embodiments, the guide nucleic acid 350 is between 20 and 35 nucleotides in length. In some embodiments, the guide nucleic acid 350 and the Argonaute protein 360 are bound in a complex 380 before contacting the RCP 340.

### B. Nuclease-Active Argonaute Proteins

In some embodiments, the method comprises contacting an RCP generated in a biological sample with an Argonaute-guide nucleic acid complex comprising a guide nucleic acid and an Argonaute protein that has cutting activity (i.e., is slicer-active). Any suitable Argonaute protein for cutting RNA in a nucleic acid duplex (e.g., within the guide target sequence bound to the guide nucleic acid) can be used. Generally, Argonaute proteins contain 6 main domains (N-terminal, L1 (Linker 1), PAZ (Piwi-Argonaute-Zwille), L2 (Linker 2), MID (Middle) and PIWI (P-element induced wimpy testis) responsible for binding of a guide nucleic acid and recognition of a guide target sequence. More specifically, the PIWI domain can possess a nuclease active site with a catalytic tetrad (e.g., amino acid sequence DEDX, wherein X is the amino acid D, H, or K), wherein the catalytic tetrad coordinates two divalent metal cations (e.g., Mn²⁺, Mg²⁺, etc.) essential for target cleavage. In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule.

In some embodiments, the Argonaute protein is a naturally-occurring protein (e.g., naturally occurs in prokaryotic or eukaryotic cells). In some embodiments, the Argonaute protein is not a naturally-occurring protein (e.g., a variant or mutant protein). In some embodiments, the Argonaute protein is a recombinant protein. In some embodiments, the Argonaute protein is genetically engineered (such as an Argonaute protein described in WO 2019/222036, the contents of which is herein incorporated by reference in its entirety). In some embodiments, the Argonaute protein is exogenous to the biological sample contacted with the probe or probe set.

In some embodiments, the Argonaute protein is a eukaryotic Argonaute protein. Generally, eukaryotic Argonaute proteins can mediate cutting of a target RNA with a guide nucleic acid of RNA. In some embodiments, an Argonaute protein is of plant, algal, fungal (e.g., yeast), or animal (e.g., human, rodent, fruit fly, cnidarian, echinoderm, nematode, fish, amphibian, reptile, bird, etc.) origin. In some embodiments, the Argonaute protein is Ago1, Ago2, Ago3, Ago4, PIWI 1, PIWIL 2, PIWI 3, or PIWI 4 (such as the Argonaute proteins described in WO 2007/048629, the content of which is herein incorporated by reference in its entirety). In some embodiments, the Argonaute protein is Ago2. In some embodiments, the Ago2 is Drosophila Ago2. In some embodiments, the Argonaute protein is a recombinant Drosophila Argonaute protein. In some embodiments, the Argonaute protein is expressed in a mammalian cell line. In some embodiments, the Argonaute protein is a Drosophila Argonaute protein expressed in a mammalian cell line. In some embodiments, a Drosophila Argonaute protein is expressed using a method such that a loading complex specific to Drosophila species is not provided to obtain guide-free proteins. In some embodiments, the Argonaute protein is a purified recombinant Drosophila Argonaute protein. In some embodiments, the Argonaute protein is expressed in an insect cell line, such as a Schneider 2 (S2) cell line. In some embodiments, the Argonaute protein is a Drosophila Argonaute protein expressed in an insect cell line, such as a S2 cell line. In some embodiments, the Drosophila Argonaute protein is loaded with the guide nucleic acid prior to contacting the biological sample. In some embodiments, the eukaryotic Argonaute protein is a mammalian Argonaute protein. In some embodiments, the mammalian Argonaute protein is selected from AGO1, AGO2, AGO3, and AGO4. In some embodiments, the mammalian Argonaute protein is a human Argonaute protein. In some embodiments, the human Argonaute protein is an AGO1, AGO3, or AGO4 protein which has been modified to have slicer activity (See Faehnle et al. The making of a slicer: activation of a human Argonaute-1. Cell Reports 2015 Jun. 27, 3(6): 1901-1909, which is herein incorporated by reference in its entirety). In some embodiments, the human Argonaute protein is an AGO3. In some embodiments, the human Argonaute protein is a human AGO2 protein (See McGeary et al., The Biochemical Basis of microRNA Targeting Efficacy. Science 2019 Dec. 20; 366(6472): eaav1741., which is herein incorporated by reference in its entirety).

In some embodiments, the Argonaute protein is a prokaryotic Argonaute protein or a variant thereof. Generally, prokaryotic Argonaute proteins can mediate cutting of a target RNA with a guide oligonucleotide. In some cases, the prokaryotic Argonaute protein uses RNA as a guide oligonucleotide. In some cases, the prokaryotic Argonaute protein uses DNA as a guide oligonucleotide. In some embodiments, the Argonaute protein is a Nitratireductor (optionally Nitratirereductor sp. XY-223), Enhydrobacter (optionally Enhydrobacter aerosaccus), Mesorhizobium (optionally Mesorhizobium sp. CNPSo 3140), Hyphomonas (optionally Hyphomonas sp. T16B2), Pseudooceanicola (optionally Pseudooceanicola lipolyticus), Tateyamaria (optionally Tateyamaria omphalii), Bradyrhizobium (optionally Bradyrhizobium sp. ORS 3257), Dehalococcoides (optionally Dehalococcoides mccartyi), Chroococcidiopsis (optionally Chroococcidiopsis cubana), Runella (optionally Runella slithyformis), Roseivirga (optionally Rosevirga seohaensis), Spirosoma (optionally Spirosoma endophyticum), Pedobacter (optionally Pedobacter yonginense, Pedobacter insulae, or Pedobacter nyackensis), Planctomycetes bacterium (optionally Planctomycetes bacterium TBKIr or Planctomycetes bacterium V6), Dyadobacter (optionally Dyadobacter sp. QTA69), Mucilaginibacter (optionally Mucilaginibacter gotjawali, Mucilaginibacter polytichastri or Mucilaginibacter paludis), Hydrobacter (optionally Hydrobacter penzbergensis), Chitinophaga (optionally Chitinophaga costaii), Cytophagaceae bacterium (optionally Cytophagaceae bacterium SJW1-29), Emticicia (optionally Emticicia oligotrophica), Runella (optionally Runella sp. YX9), or Spirosoma (optionally Spirosoma pollinicola) Argonaute protein (See Li et al., “A programmable pAgo nuclease with RNA target preference from the psychrotolerant bacterium Mucilaginibacter paludis” Nucleic Acids Res. 2022 May 20; 50(9): 5226-5238; Lisitskaya et al., “Programmable RNA targeting by bacterial Argonaute nucleases with unconventional guide binding and cleavage specificity.” Nat Commun. 2022 Aug. 8; 13(1): 4624; Sun et al., “An Argonaute from Thermus parvatiensis exhibits endonuclease activity mediated by 5′ chemically modified DNA guides.” Acta Biochim Biophys Sin (Shanghai). 2022 May 25; 54(5): 686-695; U.S. Pat. No. 10,253,311; U.S. Ser. No. 15/089,243; U.S. Ser. No. 17/575,957; U.S. Ser. No. 17/854,897; and WO 2022/222920 each of which herein incorporated by reference in their entireties). In some embodiments, the Argonaute protein is from Thermus thermophilus. In some embodiments, the Argonaute protein is from Marinitoga piezophile (See Lapinaite et al, “Programmable RNA recognition by a CRISPR-associated Argonaute.” PNAS 2018 Mar. 27; 115(13): 3368-3373, which is herein incorporated by reference in its entirety). In some embodiments, the Argonaute protein is from Rhobacter sphaeroidis (See Miyoshi et al, “Structural basis for the recognition of guide RNA and target DNA heteroduplex by Argonaute.” Nature Comm 2016; 7:11846, which is herein incorporated by reference in its entirety). In some embodiments, the Argonaute protein is from Thermomyces thermophilus (such as an Argonaute protein described in patent application no. CN202210082114, the content of which is herein incorporated by reference in its entirety). In some embodiments, an Argonaute protein is from Vanderwaltozyma polyspora (also known as Kluyveromyces polysporus) (such as an Argonaute protein described in WO 2018/112336, the content of which is herein incorporated by reference in its entirety). In some embodiments, the Argonaute protein is an Argonaute protein from Clostridium perfringens (CpAgo) or an Argonaute protein from Intestinibacter bartlettii (IbAgo) (See Cao et al, Argonaute proteins from human gastrointestinal bacteria catalyze DNA-guided cleavage of single- and double-stranded DNA at 37 C. Cell Discovery 2019 5(38), which is hereby incorporated by reference in its entirety). In some embodiments, the Argonaute protein is an Argonaute protein from Clostridium butyricum (CbAgo) (See Hegge et al. DNA-guided DNA cleavage at moderate temperatures by Clostridium butyricum Argonaute. BioRXIV 2019, and Kuzmenko et al. Programmable DNA cleavage by Ago nucleases from mesophilic bacteria Clostridium butyricum and Limnothrix rosea. BioRXIV 2019, each of which is hereby incorporated by reference in its entirety). In some embodiments, the Argonaute is a MpAgo derived from Marinitoga piezophile (MpAgo). In some embodiments, the MpAgo protein forms a complex with a guide nucleic acid to form a MpAgo-guide nucleic acid complex. In some embodiments, the guide nucleic acid of the MpAgo-guide nucleic acid complex comprises a 5′-BrdU modification that enables increased binding stability of MpAgo-guide nucleic acid complex to a target nucleic acid compared to a non-modified guide nucleic acid. In some embodiments, a seed region of the MpAgo-guide nucleic acid complex comprises nucleotides 5-15 of the guide nucleic acid. In some embodiments, full complementarity of the 5-15 nucleotide seed region of the MpAgo-guide nucleic acid to a target nucleic acid enables binding of the MpAgo-guide nucleic acid complex. In some embodiments, the MpAgo-guide nucleic acid complex can detect SNVs at the position of the target nucleic acid that is complementary to nucleic acids 6-7 of the guide nucleic acid. In some embodiments, the MpAgo-guide nucleic acid can detect modified nucleotides in the target nucleic acid at positions complementary to nucleotides 6-7 of the guide nucleic acid.

In some embodiments, the Argonaute protein is a variant of a DNA-cutting Argonaute protein. In some cases, a DNA-cutting Argonaute protein is mutated to cut an RNA substrate via selection and/or directed evolution. In some embodiments, the Argonaute is a DNA-guided Pyrococcus furiosus that cuts single- and double-stranded DNA (See Swarts et al, “Argonaute of the archaeon Pyrocuccus furiosus is a DNA-guided nuclease that targets cognate DNA.” Nucleic Acids Research Volume 43, Issue 10, 26 May 2015, Pages 5120-5129, which is herein incorporated by reference in its entirety).

In some embodiments, the Argonaute protein comprises one or more amino acid substitutions compared to any of the species of Argonaute protein described herein. In certain embodiments, the one or more amino acid substitutions are conservative substitutions. In some aspects, conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine(S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g., U.S. Pat. No. 8,562,989; pages 13-15 “Biochemistry” 2^{nd} ED. Lubert Stryer ed (Stanford University); Henikoff et al., PNAS 1992 Vol 89 10915-10919; Lei et al., J Biol Chem 1995 May 19; 270(20): 11882-6, which is herein incorporated by reference in its entirety). An amino acid substitution may include replacement of one amino acid in a polypeptide with another amino acid. Amino acid substitutions may be introduced to generate a modified Argonaute protein.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some contexts, conservative substitutions can involve the exchange of a member of one of these classes for another member of the same class. In some contexts, non-conservative amino acid substitutions can involve exchanging a member of one of these classes for another class. In some contexts, particular substitutions can be considered “conservative” or “non-conservative” depending on the stringency and context and environment of the particular residue in primary, secondary and/or tertiary structure of the protein.

In some embodiments herein is presented a method for analyzing a biological sample comprising a rolling circle amplification product (RCP), comprising contacting the biological sample with an Argonaute protein and a guide nucleic acid. In some embodiments, the Argonaute protein has slicer activity. In some embodiments, the Argonaute protein is nuclease active. In some embodiments, the Argonaute protein is capable of cutting the RCP. In some embodiments, the RCP comprises a target sequence that is a complement of the seed sequence of the guide nucleic acid, and the Argonaute protein cuts the RCP at the target sequence.

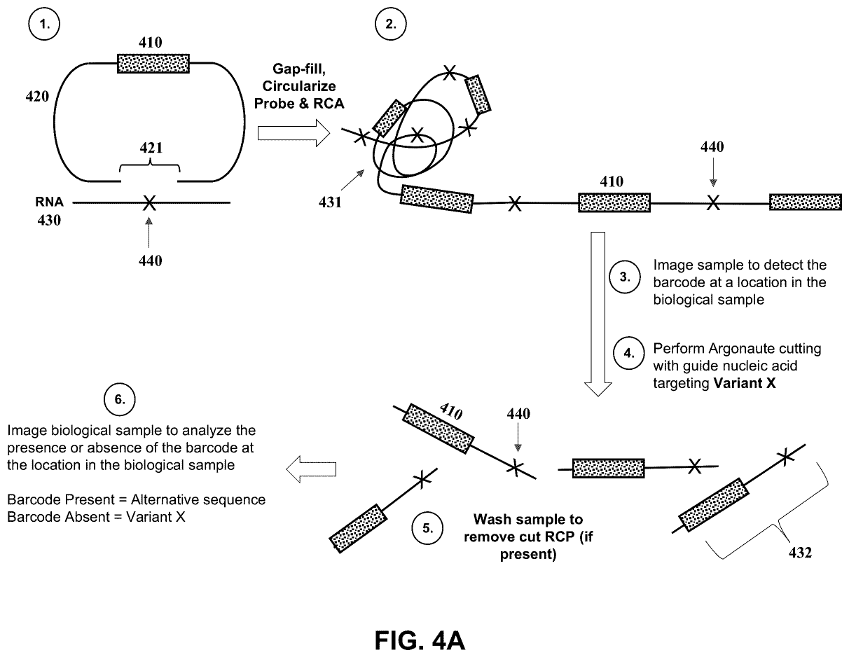

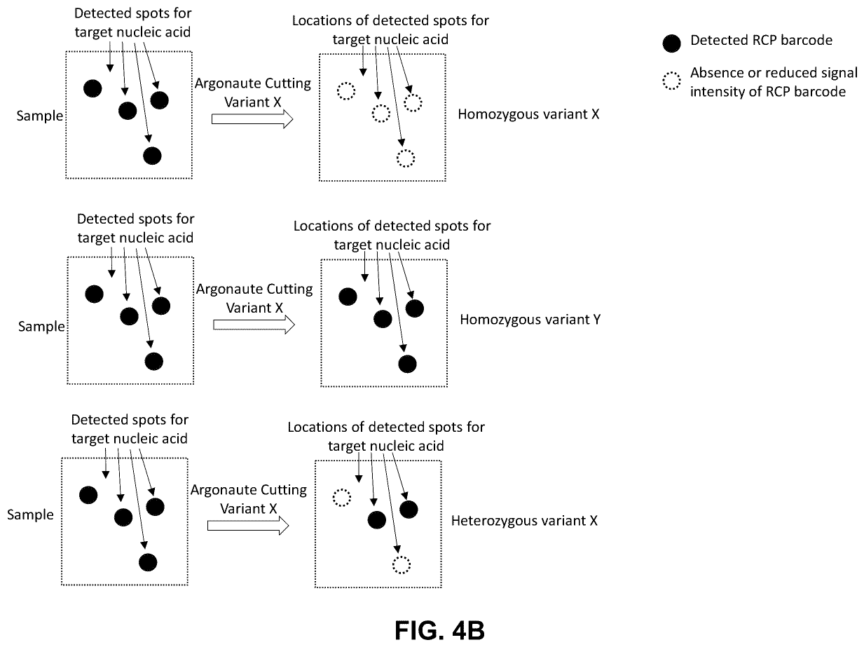

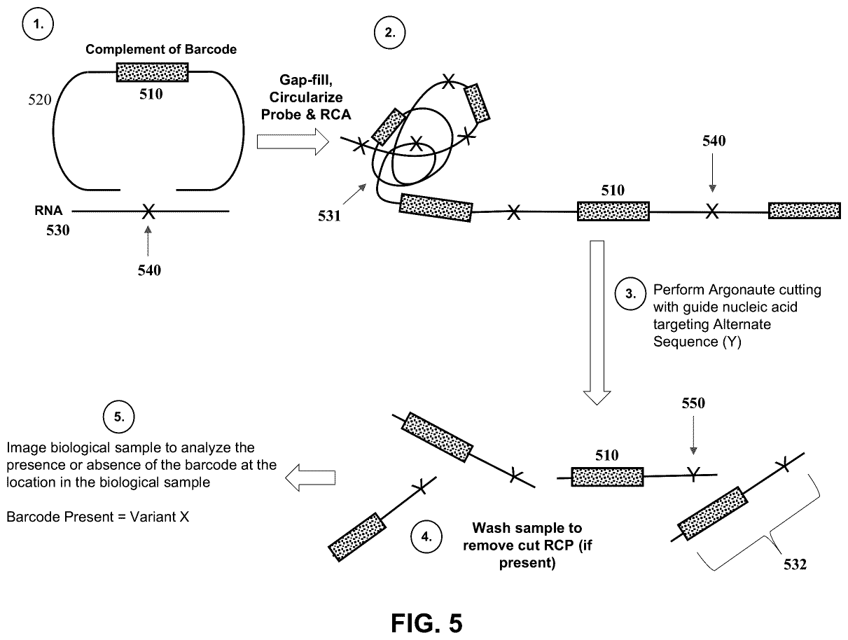

In some embodiments, provided herein is an Argonaute protein with endonuclease activity (i.e., slicer activity). In some embodiments, the Argonaute protein is capable of cutting a target nucleic acid (e.g., a target RNA or DNA). In some embodiments, the Argonaute of the Argonaute-guide nucleic acid complex is capable of cutting an RCP generated from a circularized probe or probe set in a biological sample. In some embodiments, the Argonaute protein of the Argonaute-guide nucleic acid complex is capable of cutting the variant sequence of interest, for example, as shown in FIG. 4A. In some embodiments, the Argonaute protein of the Argonaute-guide nucleic acid complex is capable of cutting the alternative variant sequence, for example, as shown in FIG. 5. In some embodiments, an Argonaute-guide nucleic acid complex comprising an Argonaute protein with endonuclease activity (i.e., slicer activity) is capable of cutting a target nucleic acid at or near a variant sequence that is complementary to the seed sequence of the guide nucleic acid. In some embodiments, cutting at or near a variant sequence means cutting within about 20 nucleotides or fewer, or cutting within about 15 nucleotides or fewer, or cutting within about 12 nucleotides or fewer, or cutting within about 10 nucleotides or fewer, or cutting within about 8 nucleotides or fewer, or cutting within about 8 nucleotides or fewer, or cutting within about 6 nucleotides or fewer, or cutting within about 4 nucleotides or fewer, or cutting within about 2 nucleotides or fewer, or cutting within about 1 nucleotide or fewer from the variant sequence on the target RCP.

In some embodiments, provided herein are RCPs generated from a gap-fill circularizable probe or probe set. In some embodiments, the 3′ end and the 5′ end of the probe or probe set are ligated without gap filling prior to ligation, for example, as shown in FIG. 3A. In some embodiments, the ligation of a 3′ end and a 5′ end of the probe or probe set is preceded by a gap-fill reaction to generate a circularized nucleic acid template for rolling circle amplification. In some embodiments, the probe or probe set comprises a first probe region and a second probe region that hybridize to a first target sequence and a second target sequence, respectively, in a target nucleic acid in the biological sample. In some embodiments, the first and second target sequences flank a gap sequence in the target nucleic acid. In some embodiments, the gap sequence comprises a variant sequence. In some embodiments, the 3′ end and the 5′ end of the probe or probe set are ligated without gap filling prior to ligation. In some embodiments, the rolling circle amplification product that is cut by the Argonaute protein is generated from a circularized nucleic acid template that hybridizes to a nucleic acid having high expression in the biological sample. Optionally, in some embodiments the nucleic acid that has high expression is a nucleic acid that can be detected at a mean count of more than 20 transcripts per cell for at least a subset of cells in the biological sample.

In some embodiments, the Argonaute protein possesses nuclease activity in a buffer comprising divalent cations. In some embodiments, the cutting of the guide target sequence by the Argonaute-guide nucleic acid complex is performed in a buffer comprising at least one divalent cation (e.g., Fe²⁺, Co²⁺, Ni²⁺, Cu²⁺, Zn²⁺, Mg²⁺, Mn²⁺, or Ca²⁺). In some embodiments, the cutting of the guide target sequence by the Argonaute-guide nucleic acid complex is performed in a buffer comprising Mg²⁺ and/or Mn²⁺. In some embodiments, the Argonaute protein possesses nuclease activity at a certain temperature and cutting of the guide target sequence by the Argonaute-guide nucleic acid complex is performed at the temperature at which the Argonaute protein has cleavage activity.

In some embodiments, the method further comprises incubating the biological sample with the Argonaute and the guide nucleic acid at a temperature between 20° C. and 50° C. to allow the cutting of the RCP by the Argonaute. Optionally, in some embodiments the temperature is between 30° C. and 44° C. In some embodiments, the incubating occurs in a buffer comprising Mg²⁺ and/or Mn²⁺ cations. In some embodiments, the method further comprises washing the biological sample. Optionally, in some embodiments, the washing is performed under less than stringent conditions.

### E. Argonaute-Mediated Obliteration of RCPs

In some embodiments, the method presented herein further comprises cutting the RCP with the Argonaute-guide nucleic acid complex at the site of a variant sequence (e.g., at the variant sequence of interest, or at an alternative variant sequence). In some embodiments, the Argonaute-guide nucleic acid complex cuts the RCP at a variant sequence of interest, for example, as shown in FIG. 4A. In some embodiments, the Argonaute-guide nucleic acid complex cuts the RCP at an alternative variant sequence, for example, as shown in FIG. 5. In some embodiments, the rolling circle amplification product (RCP) cut by the Argonaute-guide nucleic acid complex is a rolling circle amplification product of a plurality of rolling circle amplification products generated from a circularized probe or probe set associated with a target nucleic acid in a biological sample. In some embodiments, at least one rolling circle amplification product of the plurality of rolling circle amplification products is cut by the Argonaute protein. In some embodiments, at least one rolling circle amplification product of the plurality of rolling circle amplification products is not cut by the Argonaute protein. In some embodiments, the rolling circle amplification product that is cut by the Argonaute protein comprises multiple copies of the variant sequence of interest. In some embodiments, the rolling circle amplification product that is cut by the Argonaute protein comprises multiple copies of the alternative variant sequence.

FIG. 4A shows an embodiment of a method for detecting the presence or absence of a variant sequence of interest X 440 at a location in a biological sample. RCPs comprising multiple copies of the variant sequence of interest or an alternative variant sequence are generated from gap-fill padlock probes 420 comprising a barcode region 410, wherein the probes bind to a target nucleic acid 430 such as an mRNA such that the binding sites flank the variant sequence 440 such that there is a gap 421. The gap 421 in the padlock probe 420 is filled (either via splint oligonucleotide ligation or via polymerization; see Section IV) to generate a gap-filled probe that is circularized and amplified via rolling circle amplification to generate a rolling circle amplification product (RCP) 431 comprising multiple copies of the variant sequence of interest 440 and/or an alternative variant sequence, and multiple copies of the barcode region 410. A first round of detectably labeled probes capable of hybridizing to the barcode region are contacted with the sample for a first round of imaging. After the first round of imaging, the biological sample is washed to remove the first round of detectably labeled probes. Argonaute-guide nucleic acid complexes comprising a nuclease-active Argonaute protein and a guide nucleic acid comprising a sequence complementary to the variant sequence of interest X are then contacted with the biological sample. In some embodiments, the Argonaute-guide nucleic acid complexes are incubated with the RCPs, allowing cutting of the RCPs by the Argonaute protein at the site of the variant sequence of interest X. The cutting of the RCPs at the site of the variant sequence of interest X results in cut RCP fragments 432 comprising a barcode sequence 410 and the variant sequence of interest X 440. The biological sample is then washed to remove the cut RCP fragments 432. In some embodiments, a second round of detectably labeled probes capable of hybridizing to the barcode region are contacted with the sample for a second round of imaging. Results from the two rounds of imaging are compared, such that the presence of the barcode at a location across two rounds of imaging indicates the presence of an alternative sequence at the location in the biological sample, and the absence of the barcode at a location in the second round of imaging indicates the presence of the variant sequence of interest X at the location in the biological sample.

In some embodiments, the Argonaute protein cleaves an RCP at a variant sequence that is hybridized to a complementary region of the guide nucleic acid. In some embodiments, the Argonaute cleaves the RCP bound to the guide nucleic acid between any two positions between position 9 and 12 of the guide nucleic acid. In some embodiments, the Argonaute cleaves the RCP at a position between positions 11 and 12 or between positions 10 and 11 of the guide nucleic acid.

In some embodiments, the Argonaute protein cuts the RCP at or near the variant sequence. In some embodiments, at or near means that the cutting occurs within about 12 or fewer nucleotides, within about 10 or fewer nucleotides, within about 8 or fewer nucleotides, within about 6 or fewer nucleotides, within about 4 or fewer nucleotides, within about 2 or fewer nucleotides, or within about 1 or fewer nucleotides, of the variant sequence (e.g., the wildtype or mutant SNV).

In some embodiments, the Argonaute-guide nucleic acid complex cuts an RCP at a variant sequence of interest or at an alternative sequence. In some embodiments, the Argonaute of the Argonaute-guide nucleic acid complex cuts the RCP at the variant sequence of interest. In some embodiments, the Argonaute of the Argonaute-guide nucleic acid complex cuts the RCP at an alternative sequence. A schematic illustration of Argonaute-mediated cutting of the RCP at the alternative sequence of interest is shown in FIG. 5. In some embodiments, the cutting of RCP by the Argonaute of the Argonaute-guide nucleic acid complex is performed in a buffer comprising Mg²⁺ and/or Mn²⁺ cations. In some embodiments, optionally, the cutting of the RCP by the Argonaute of the Argonaute-guide nucleic acid complex is performed in a buffer comprising Mg²⁺ cations.

In some embodiments, the method further comprises incubating the biological sample with the Argonaute and the guide nucleic acid at a temperature between 20° C. and 50° C. to allow the cutting of the RCP by the Argonaute. Optionally, in some embodiments the temperature is between 30° C. and 44° C. In some embodiments, the method further comprises incubating the biological sample with the Argonaute and the guide nucleic acid at a temperature of at least 65° C. to allow the cutting of the RCP by the Argonaute. In some embodiments, the method further comprises washing the biological sample, for example, as shown in FIG. 5. Optionally, in some embodiments, the washing is performed under less than stringent conditions. In some embodiments, the washing removes cut RCPs 532 from the biological sample.

FIG. 5 shows an embodiment of a method for detecting the presence or absence of a variant sequence of interest X 540 at a location in a rolling circle amplification product 531 generated in a biological sample, using Argonaute-mediated obliteration of an alternative sequence Y 550. A padlock probe 520 comprising a complement of a barcode 510 hybridizes to target regions on either side of a variant sequence of interest X 540 in a target nucleic acid 530 (e.g., an RNA). The padlock probe is gap-filled, circularized, and amplified via rolling circle amplification to generate a rolling circle amplification product 531 comprising multiple complements of the barcode 510 and multiple copies of the variant sequence of interest X 540 and/or the alternative sequence Y 550. The RCPs are contacted with slicer-active Argonaute-guide nucleic acid complexes with guide nucleic acids targeting alternative sequence Y 550. The Argonaute-guide nucleic acid complexes cut the RCPs 531 containing the alternative sequence of interest Y 540 to generate cut RCPs 532 that can then be removed from the biological sample via washing. Intact RCPs 351, comprising multiple copies of the complement of the barcode 510 and multiple copies of the variant sequence of interest X 540, are imaged to analyze the presence or absence of the barcode 510 at the location in the biological sample. The presence of the barcode 510 indicates the presence of the variant sequence of interest X 540 at the location in the biological sample.

In some embodiments, the method further comprises imaging the biological sample to analyze the presence or absence of the barcode sequence at the location in the biological sample, thereby analyzing the presence or absence of the variant sequence of interest. In some embodiments, imaging the biological sample comprises imaging fluorescent puncta at a location in the biological sample at two separate time points. FIG. 4B shows an example of a decoding scheme for analyzing the presence or absence of the barcode sequence at a location in the biological sample across two time points based on imaging signals from fluorescent puncta. In some embodiments, analyzing the biological sample comprises comparing imaging results from two rounds of imaging performed on the biological sample. In some embodiments, Argonaute-mediated obliteration of a variant sequence or an alternative sequence is performed between the two rounds of imaging. In some embodiments, absence or relative absence (e.g., low levels or decreased levels of signal relative to a set threshold indicating presence of the detectable label, or absence of signal) of the signal from the detectable label associated with the target nucleic acid sequence at the location in the biological sample upon reimaging indicates the presence of the variant sequence at the location in the biological sample. In some embodiments, absence or relative absence (e.g., low or decreased levels of signal relative to a set threshold indicating presence of the detectable label, or absence of signal) of the signal from the detectable labels associated with the target nucleic acid sequence at the location in the biological sample may indicate the presence of the homozygous variant of interest (e.g., homozygous variant “X”). In some embodiments, presence or increased presence (e.g., a high level of signal or an increased level of signal, optionally wherein the level of signal is at or above a set threshold indicating presence of the detectable label) of the signal from the detectable label associated with the target nucleic acid sequence at the location in the biological sample upon reimaging indicates the absence of the variant sequence at the location in the biological sample. In some embodiments, presence or increased presence (e.g., a high level of signal or an increased level of signal, optionally wherein the level of signal is at or above a set threshold indicating presence of the detectable label) of the signal from the detectable labels associated with the target nucleic acid sequence at the location in the biological sample may indicate the absence of the homozygous variant of interest. In some embodiments, presence or increased presence (e.g., a high level of signal or an increased level of signal, optionally wherein the level of signal is at or above a set threshold indicating presence of the detectable label) of the signal from the detectable labels associated with the target nucleic acid sequence at the location in the biological sample may indicate the presence of an alternative homozygous (e.g., homozygous variant “Y”). In some embodiments, partial loss of the signal from the detectable label associated with the target nucleic acid sequence at the location in the biological sample upon reimaging indicates the presence of a heterozygous variant of interest (e.g., heterozygous variant “XY”). In some embodiments, loss of signals from approximately half of detected spots from the detectable label associated with the target nucleic acid sequence at the location in the biological sample upon reimaging indicates the presence of a heterozygous variant of interest (e.g., heterozygous variant “XY”).

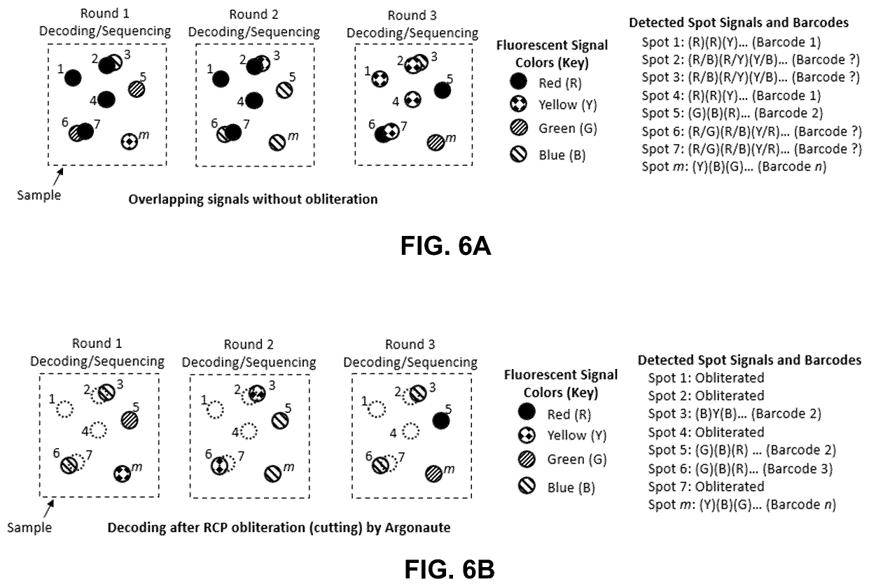

In some embodiments, Argonaute-mediated obliteration of alternative sequences may be useful for reducing optical crowding during imaging. For example, FIG. 6A illustrates a scenario in which optical crowding from detectable fluorophores at a location in the biological sample results in decreased ability to decode the signal, due to overlapping signals at the location in the biological sample. FIG. 6B shows the same detecting and decoding scheme as in FIG. 6A after the use of Argonaute-mediated obliteration to reduce the number and/or density of detectable molecules present at a given location in the biological sample, thus enabling greater decoding efficiency of the detectable molecules still present at the location in the biological sample following obliteration of a defined subset. In some embodiments, Argonaute-mediated obliteration is used to increase the ability to decode the signal by removing overlapping optical signals at the same spatial location in the biological sample. In some embodiments, the Argonaute-guide nucleic acid complex cuts a plurality of RCPs that share a common sequence. In some embodiments, a plurality of Argonaute-guide nucleic acid complexes cut a plurality of RCPs associated with different target nucleic acids.

In some embodiments, the method comprises detecting a rolling circle amplification product that has not been cut by the Argonaute protein (e.g., detecting the rolling circle amplification product at a location in the biological sample). In some embodiments, the method comprises comparing detected RCPs prior to and after cutting by the Argonaute proteins. In some embodiments, the method comprises comparing detected signals at particular locations in the biological sample prior to and after cutting by the Argonaute proteins. In some embodiments, the method comprises analyzing the presence or absence of a rolling circle amplification product that has not been cut by the Argonaute protein. In certain embodiments, the presence or absence of the rolling circle amplification product is analyzed after incubating the biological sample with the nuclease active Argonaute protein and guide nucleic acid.

In some embodiments, detecting or analyzing the presence or absence of the rolling circle amplification product is performed using a detectably labeled nuclease-deficient Argonaute and guide nucleic acid complex. In some embodiments, detecting or analyzing the presence or absence of the rolling circle amplification product comprises sequential hybridization of probes, e.g., sequencing by hybridization and/or sequential in situ fluorescence hybridization. Sequential fluorescence hybridization can involve sequential hybridization of the detectably labeled probes disclosed herein. In some embodiments, a method disclosed herein comprises sequential hybridization of detectably labeled probes and intermediate probes that are not detectably labeled per se but are capable of binding (e.g., via nucleic acid hybridization) and being detected by detectably labeled probes, such as fluorescently labeled probes. Exemplary methods comprising sequential fluorescence hybridization of probes are described in US 2019/0161796, US 2020/0224244, US 2022/0010358, US 2021/0340618, WO 2020/123742, and WO 2021/138676, each of which is incorporated herein by reference in its entirety.

In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting comprises binding an intermediate probe directly or indirectly to the RCP, binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized probe or probe set as a template (e.g., as described in Section IV). In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method comprises performing one or more wash steps to remove unbound and/or nonspecifically bound intermediate probe molecules from the primary probes or the products of the primary probes.

In some embodiments, the detecting comprises: detecting signals associated with detectably labeled probes that are hybridized to barcode regions or complements thereof in the primary probe or probe set or a product thereof (e.g., an RCP); and/or detecting signals associated with detectably labeled probes that are hybridized to intermediate probes which are in turn hybridized to the barcode regions or complements thereof. In some embodiments, the detectably labeled probes is fluorescently labeled.

In some embodiments, the methods comprise detecting the sequence in all or a portion of an RCP, such as one or more barcode sequences present in the probe or probe set or RCP. In some embodiments, the sequence of the RCP, or barcode thereof, is indicative of a sequence of the target nucleic acid. In some embodiments, the analysis and/or sequence determination comprises detecting a sequence in all or a portion of the RCP and/or in situ hybridization to the RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises hybridizing to a probe directly or indirectly a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample.

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, each of which is herein incorporated by reference in its entirety. In addition, examples of methods and systems for performing in situ sequencing are described in US 2016/0024555, US 2019/0194709, and in U.S. Pat. Nos. 10,138,509, 10,494,662 and 10,179,932, each of which is herein incorporated by reference in its entirety.

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, each of which is herein incorporated by reference in its entirety.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes comprise fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step can comprise contacting the biological sample with one or more detectably labeled probes that directly or indirectly hybridize to the barcode sequences or complements thereof (e.g., in amplification products generated using the probes or probe sets), and dehybridizing the one or more detectably labeled probes. In any of the embodiments herein, the contacting and dehybridizing steps is repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting step can comprise contacting the biological sample with one or more first detectably labeled probes that directly hybridize to the RCPs. In some instances, the detecting step can comprise contacting the biological sample with one or more first detectably labeled probes that indirectly hybridize to the RCPs. In any of the embodiments herein, the detecting step can comprise contacting the biological sample with one or more first detectably labeled probes that directly or indirectly hybridize to the RCPs.

In any of the embodiments herein, the detecting step can comprise contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In any of the embodiments herein, the detecting step can further comprise dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In any of the embodiments herein, the contacting and dehybridizing steps are repeated with the one or more intermediate probes, the one or more detectably labeled probes, one or more other intermediate probes, and/or one or more other detectably labeled probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597, each of which is herein incorporated by reference in its entirety.

In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), each of which is herein incorporated by reference in its entirety.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, each of which is herein incorporated by reference in its entirety.

### IV. Gap-filled Probes and Probe Circularization

In some embodiments, a probe or probe set is designed to hybridize to a target nucleic acid (e.g., a target RNA) around (e.g., flanking by binding an upstream sequence and a downstream sequence) a variant nucleotide or short variant sequence (such as an SNV or a mutation hotspot), leaving a gap of one or more nucleotides. In some embodiments, a gap is filled by extending the 3′ end of a probe which is a gap-fill padlock probe. In some embodiments, filling a gap comprises extending the 3′ end of the first probe over the targeted variant nucleotide or short variant sequence (e.g., an SNP or SNV), incorporating the sequence information of the correct nucleotide(s) in the form of complementary nucleotide(s) in the gap-filled probe, and ligating the gap-filled probe to generate a circularized probe. In some embodiments, the circularized probe is amplified, producing an RCA product containing a plurality of unit sequences each comprising a copy of the variant nucleotide or short variant sequence.

In some embodiments, provided herein are probe molecules that are circularized after hybridization to a target nucleic acid and gap-filling using the target nucleic acid as a template. In some embodiments, a probe or probe set is hybridized to a target nucleic acid molecule comprising a gap sequence which comprises one of multiple variant sequences, and the probe or probe set is gap-filled and then circularized to generate a circularized probe comprising a gap-filled sequence complementary to the gap sequence in the target nucleic acid molecule. In some embodiments, the circularized probe comprising at least portions of the complement of the gap sequence is amplified (e.g., through RCA) and the amplification product is detected in order to detect the variant sequence in the target nucleic acid molecule.

In some embodiments, herein is a method for analyzing a biological sample comprising contacting the biological sample with a gap-fill circularizable probe or probe set (e.g., a gap-fill padlock probe or a gap-fill circularizable probe set). In some embodiments, the circularizable probe or probe set comprise a barcode sequence. In some embodiments, the probe or probe set comprises a first probe region and a second probe region that hybridize to a first target sequence and a second target sequence, respectively, in a target nucleic acid in the biological sample. Examples of gap-fill circularizable probes or probe sets are shown in FIGS. 2 201, 221, 231, and 241 and FIG. 3A 301-302. In some embodiments, the first and second target sequences flank a gap sequence in the target nucleic acid. In some embodiments, the gap sequence comprises a region of interest comprising a variant sequence of a plurality of possible variant sequences. In some embodiments, the gap sequence comprises a region of interest comprising either a variant sequence of interest or an alternative sequence. In some embodiments, the gap sequence comprises a variant sequence. In some embodiments, the gap sequence comprises a mutant sequence. In some embodiments, the gap sequence comprises a wild-type sequence. In some embodiments, the gap sequence is a single nucleotide.

In some embodiments, provided herein is a method for analyzing a biological sample, the method comprising contacting the biological sample with a probe or probe set comprising a first probe region and a second probe region (e.g., 5′ and 3′ arms of a padlock probe) that hybridize to a first target sequence and a second target sequence, respectively, in a target nucleic acid (e.g., an RNA or cDNA) in the biological sample. In some embodiments, the first and second probe regions of the probes or probe sets are common among a plurality of probes or probe sets that target a plurality of target nucleic acids that comprise different variant sequences. In some embodiments, a probe or probe set comprises a sequence that is complementary to two or more target nucleic acids (e.g., target RNAs) that comprise different variant sequences. In some embodiments, each of the plurality of target nucleic acids comprises a common first target sequence (among the plurality of target nucleic acids) and a common second target sequence (among the plurality of target nucleic acids) that are complementary to the common first and second probe regions, respectively, among the plurality of probes or probe sets. In some embodiments, the plurality of probes or probe sets comprise molecules of the same nucleic acid sequence. In some embodiments, the plurality of probes or probe sets comprise molecules of different nucleic acid sequences. In some embodiments, any two or more different nucleic acid sequences of the probes or probe sets comprise i) the common first and second probe regions, and ii) different backbone sequences that are not complementary to target nucleic acid sequences. The different backbone sequences may each comprise a different barcode region. In some embodiments, the barcode region may identify one or more probes or probe sets from other probes or probe sets. In some embodiments, the barcode sequence is associated with, corresponds to, and/or identifies a target nucleic acid or a sequence therein. In some embodiments, the barcode sequence is associated with, corresponds to, and/or identifies the plurality of target nucleic acids that comprise different variant sequences (e.g., a barcode sequence can correspond to target nucleic acids of a gene comprising various SNPs and/or point mutations in the gene).

In some embodiments, the first and second target sequences are separated by a gap sequence in the target nucleic acid. In some embodiments, the gap sequence is about or at least 4, about or at least 6, about or at least 8, about or at least 10, about or at least 12, about or at least 14, about or at least 16, about or at least 18, about or at least 20, or more nucleotides in length. In some embodiments, the gap sequence is between about 1 and 5 nucleotides in length. In some embodiments, the gap sequence is between about 1 and 3 nucleotides in length. In some embodiments, the gap is a single nucleotide. In some embodiments, the gap sequence comprises a variant sequence among a plurality of different variant sequences. In some embodiments, the plurality of probes or probe sets that target a plurality of target nucleic acids that comprise different variant sequences do not hybridize to the gap sequences (which comprise the different variant sequences), and instead hybridize to common first and second target sequences that flank the gap sequences.

In some embodiments, upon hybridization to the target nucleic acid, the probe or probe set is circularized to generate a circularized probe comprising a gap-filled region complementary to the gap sequence. The gap-filled region can be generated using gap-filling by polymerization, or gap-fill splint ligation, or a combination thereof. In some embodiments, an RCP of the circularized probe is generated in the biological sample, and the RCP comprises multiple copies of the gap sequence.

In some embodiments, the probe or probe set comprises a 5′ region and a 3′ region that hybridize to sequences adjacent to a hotspot for mutation in the target nucleic acid. In some embodiments, upon hybridization of a probe or probe set to the target nucleic acid molecule, the 3′ terminal nucleotide and the 5′ terminal nucleotide of the probe or probe set are not juxtaposed directly next to each other; as such, a ligase alone cannot catalyze the formation of a phosphodiester bond directly between the 5′ phosphate of the 5′ terminal nucleotide and the 3′ hydroxyl of the 3′ terminal nucleotide. In some embodiments, upon hybridization of a probe or probe set to the target nucleic acid molecule, the 3′ terminal nucleotide and the 5′ terminal nucleotide of the probe or probe set are separated from each other by a gap of between about 1 and about 40 nucleotides in length. In some embodiments, the gap is about 2, about 5, about 10, about 15, about 20, about 25, about 30, about 35, about 40, about 45, about 50, or of any integer (or range of integers) of nucleotides in between the indicated values. In some embodiments, the gap is no more than about 40 nucleotides in length. In some embodiments, the gap is no more than about 30 nucleotides in length. In some embodiments, the gap is about 6, about 8, about 10, about 12, about 14, about 16, about 18, about 20, about 22, about 24, or of any integer (or range of integers) of nucleotides in between the indicated values. In some embodiments, the gap is no more than about 10 nucleotides in length. In some embodiments, the gap is about 5 nucleotides in length.

In some embodiments, a probe or probe set disclosed herein does not comprise any nucleic acid barcode sequence. In some embodiments, the probes or probe sets for hybridizing to multiple different target nucleic acids can comprise a common sequence that is not complementary to the target nucleic acids. For instance, the backbone sequences of a plurality of probes or probe sets for detecting different variant sequences of a target nucleic acid can be a common backbone sequence. In other examples, the backbone sequences of a plurality of probes or probe sets for detecting different target nucleic acids comprises a common backbone sequence, and the arms of the gap-fill padlock probes is different such that they can specifically hybridize to the target nucleic acids. In some embodiments, the backbone sequences of the plurality of probes or probe sets do not contain any nucleic acid barcode sequence that uniquely corresponds to a particular target nucleic acid or a particular sequence variant thereof.

In some embodiments, a probe or probe set disclosed herein comprises one or more barcode regions. In some embodiments, each barcode region independently comprises one or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of probes or probe sets is less than the number of distinct targets of the probes or probe sets, and yet the distinct targets may still be uniquely identified from one another, e.g., by encoding a probe with a different combination of barcode sequences. However, not all possible combinations of a given set of barcode sequences need be used. For instance, each probe or probe set may contain 1, 2, 3, 4, 5, 6, 7, 8, 9, 10, 11, 12, 13, 14, 15, etc. or more barcode sequences. In some embodiments, a population of probes or probe sets may each contain the same number of barcode sequences, although in other cases, there may be different numbers of barcode sequences present on the various probes or probe sets. In some embodiments, the barcode sequences or any subset thereof in the population of probes or probe sets are independently and/or combinatorially detected and/or decoded.

In some embodiments, the method further comprises performing a gap-fill reaction on the probe or probe set to generate a gap-filled probe or probe set, for example, as shown in FIG. 2 with a gap-filled region 222 and 242 or FIG. 3A. In some embodiments the gap sequence comprises a genetic hotspot. Optionally, in some embodiments the gap sequence comprises two or more hotspot mutations. In some embodiments, the gap sequence comprises a variant sequence of a plurality of variant sequences. In some embodiments, the gap sequence comprises a variant allele of interest. In some embodiments, the gap sequence comprises a mutant allele or a wild type allele.

FIG. 3A depicts two embodiments of methods for gap-filling a padlock probe or split probe set to generate a gap-filled probe that are ligated to generate a circularized probe. A padlock probe or split probe set 301, 302 with optional ligation sites 307, 308 has a first probe sequence 303 complementary to a first target sequence 312 and a second probe sequence 304 complementary to a second target sequence 314 flanking a gap sequence 313 in a target nucleic acid 311. The gap sequence 313 contains a variant sequence of interest X 390, comprising one or more nucleotides. The probe or probe set 301, 302 hybridizes to the target sequences 312, 314 flanking gap sequence 313 containing the variant sequence of interest 390. Subsequently, in some embodiments, the gap is filled via splint oligonucleotides to generate a gap-filled probe region 305 containing a complement of the variant sequence of interest, X′ 391. In other embodiments, a polymerase is used to generate an extended probe region 306 comprising a sequence X′ 391 complementary to the variant sequence of interest X 390, thus generating a gap-filled probe.

### A. Gap-Fill Polymerization

In some embodiments, a gap in a probe or probe set hybridized to the target nucleic acid molecule may be filled by extending a 3′ end of the probe or probe set. In some embodiments, an enzyme (e.g., a polymerase) is used to extend the 3′ end using the target nucleic acid molecule as a template, thereby filling the gap using nucleotide sequence in the target nucleic acid molecule. In some embodiments, gap filling by the polymerase incorporates nucleotides residues into the probe or probe set, and the incorporated nucleotide sequence is complementary to the gap sequence or a portion thereof in the target nucleic acid molecule.

In some embodiments, performing the gap-fill reaction comprises using a gap-fill polymerase (e.g., DNA polymerase) to extend an end of the probe or probe set using the target RNA as a template to generate an extended probe, for example, as shown in FIG. 3A. In some embodiments, the extended probe is ligated to another end of the probe or probe set. In some embodiments, the gap-fill polymerase has no or little strand displacement activity. In some embodiments, the gap-fill polymerase incorporates one or more deoxyribonucleotide residues and/or one or more ribonucleotide residues into a 3′ end of the probe or probe set to generate the extended probe, optionally wherein the extended probe comprises one or more ribonucleotide residues at and/or near its 3′ end.

In some instances, the gap filling is performed using a polymerase (e.g., DNA polymerase) in the presence of appropriate dNTPs and other cofactors, under isothermal conditions or non-isothermal conditions. Exemplary DNA polymerases include but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. In some instances, the gap filling is performed using a ligase. For example, a ligase is capable of filling a gap of about 1 to 5 nucleotides. In some embodiments, the ligase is a T4 RNA ligase. In some embodiments, the ligase is a T4 DNA ligase.

In some instances, the gap filling is performed using a DNA polymerase capable of incorporating at least about 25, at least about 50, at least about 100, at least about 125, at least about 150, at least about 175, at least about 200, at least about 225, at least about 250, at least about 300, at least about 400, at least about 500, at least about 600, or at least about 1,000 nucleotides in a single binding event before dissociating from the target nucleic acid molecule. In some instances, the gap filling is performed using a DNA polymerase to fill a gap of no more than about 25, no more than about 20, no more than about 15, no more than about 10, or no more than about 5 nucleotides.

Incorporation of the correct nucleotides to a growing strand of DNA, as determined by the template, is known as sequence fidelity. In some embodiments, a high fidelity DNA polymerase is used for gap filling and examples include but are not limited to: Taq DNA polymerase, Phusion® High-Fidelity DNA Polymerase, KAPA Taq, KAPA Taq HotStart DNA Polymerase, KAPA HiFi, and/or Q5® High-Fidelity DNA Polymerase.

In some instances, the gap filling is performed using a polymerase having no or limited strand displacement activity, such that an extended 3′ region of the probe or probe set does not displace the 5′ region hybridized to the nucleic acid molecule. For example, T4 and T7 DNA Polymerases lack strand displacement activity and can be used for this purpose. In some embodiments, especially where the target nucleic acid is RNA, the polymerase can be a reverse transcriptase. Reverse transcriptases having reduced strand displacement activity can be used, see, e.g., Martín-Alonso et al., ACS Infect. Dis. 2020, 6, 5, 1140-1153, which is incorporated herein by reference in its entirety.

In some embodiments, the 3′ region of the probe or probe set extended by the enzyme (e.g., the polymerase) can be juxtaposed to the 5′ region of the probe or probe set, forming a nick. In some embodiments, the ligation involves template dependent ligation, e.g., using the gap sequence in the target nucleic acid as template. In some embodiments, the ligation involves template independent ligation. The nick can be ligated using chemical ligation. In some embodiments, the chemical ligation involves click chemistry.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. In some aspects, the ligase used herein is a DNA ligase. In some aspects, the ligase used herein is an ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD-i-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a SplintR® ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase is a ssDNA ligase. In some embodiments, the ssDNA ligase is a bacteriophage TS2126 RNA ligase or an archaebacterium RNA ligase or a variant or derivative thereof. In some embodiments, the ligase is Methanobacterium thermoautotrophicum RNA ligase 1, CircLigase™ I, CircLigase™ II, T4 RNA ligase 1, or T4 RNA ligase 2, or a variant or derivative thereof. In some embodiments, the ligase is a recombinantly-produced ligase isolated or derived from Acanthocystis turfacea chlorella virus 1 (ATCV-1) (also referred to as “LigAT”), e.g., as described in WO2022/216688, also published as US2024/0150745A1, which is herein incorporated by reference in its entirety.

### B. Gap-Fill Oligonucleotide Hybridization

In some embodiments, a gap in a probe or probe set hybridized to the target nucleic acid molecule may be filled by a splint oligonucleotide, for example, as shown in FIG. 3A. In some embodiments, the splint oligonucleotide is ligated to the probe or probe set at the 3′ and 5′ ends of the splint oligonucleotide using the target nucleic acid molecule as a ligation template. In some embodiments, the gap-filling of the probe or probe set with the splint oligonucleotide allows the gap-filled probe or probe set to subsequently be circularized to form a circularized probe comprising the gap-filled region comprising a complementary sequence to a gap sequence of the target nucleic acid. In some embodiments, the circularized probe can be amplified (e.g., with RCA) to form RCPs comprising multiple copies of the complementary sequence to the gap sequence of the target nucleic acid.

In some embodiments, performing the gap-fill reaction comprises contacting the biological sample with a library of splint oligonucleotides, for example, as shown in FIG. 3A. In some embodiments, each splint oligonucleotide comprises i) ligatable ends and ii) a hybridization region complementary to one of a plurality of different sequences. In some embodiments, the splint oligonucleotide comprises a 3′ hydroxyl group and a 5′ phosphate group. Optionally, in some embodiments, the splint oligonucleotide comprises one or more ribonucleotide residues at and/or near its 3′ end and/or a 5′ flap configured to be cleaved by a structure-specific endonuclease. In some embodiments, the variant sequence is at the 3′ or 5′ end of the gap sequence, and/or the sequence complementary to the variant sequence is at the 5′ or 3′ end of the splint oligonucleotide. In some embodiments, the variant sequence is at least 1, 2, 3, 4, 5, 6, 7, 8, 9, or more phosphodiester bonds from the 3′ or 5′ end of the gap sequence, and/or the sequence complementary to the variant sequence is at least 1, 2, 3, 4, 5, 6, 7, 8, 9, or more phosphodiester bonds from the 5′ or 3′ end of the splint oligonucleotide. Optionally, in some embodiments, the variant sequence is at or near the central nucleotide(s) of the gap sequence and/or the sequence complementary to the variant sequence is at or near the central nucleotide(s) of the splint oligonucleotide. In some embodiments, the splint oligonucleotide and/or the gap sequence is between about 2 and about 40 nucleotides in length. In some embodiments, the library of splint oligonucleotides comprises at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different hybridization region sequences. In some embodiments, the molar concentration of the library of splint oligonucleotides is about equal to or about 2, about 4, about 8, about 10, or more times the molar concentration of the probe or probe set. In some embodiments, the method comprising washing the biological sample after contacting with the library of splint oligonucleotides. In some embodiments, optionally the washing is performed under less than stringent conditions.

In some embodiments, the splint oligonucleotide comprises a sequence complementary to a nucleotide variation, a nucleotide polymorphism, a mutation, a substitution, an insertion, a deletion, a translocation, a duplication, an inversion, and/or a repetitive sequence, for identifying a variant sequence among a plurality of different sequences in situ in a biological sample. In some embodiments, the splint oligonucleotide comprises a sequence complementary to a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the splint oligonucleotide comprises a sequence complementary to a sequence comprising multiple nucleotides, and each nucleotide can be independently at the position of an SNV, an SNP, a point mutation, a single nucleotide substitution, a single nucleotide insertion, or a single nucleotide deletion. In some embodiments, the splint oligonucleotide comprises a sequence complementary to a variant sequence of a plurality of possible variant sequences, for instance, such as a variant sequence of interest or an alternative variant sequence. In some embodiments, the splint oligonucleotide comprises a sequence complementary to a mutant or wildtype variant, or a major or minor variant.

In some embodiments, provided herein is a library of splint oligonucleotides comprising i) a splint oligonucleotide comprising a sequence complementary to a nucleotide variation, a nucleotide polymorphism, a mutation, a substitution, an insertion, a deletion, a translocation, a duplication, an inversion, and/or a repetitive sequence, and ii) another splint oligonucleotide which does not comprise a sequence complementary to the nucleotide variation, nucleotide polymorphism, mutation, substitution, insertion, deletion, translocation, duplication, inversion, and/or repetitive sequence. In some embodiments, the library of splint oligonucleotides comprises i) a splint oligonucleotide comprising a sequence complementary to a variant sequence or deletion or insertion, and ii) another splint oligonucleotide which does not comprise a sequence complementary to the variant sequence or deletion or insertion. For example, wildtype and variant splint oligonucleotides in the library, when contacted with the biological sample, can compete with one another for hybridization to a gap sequence comprising a variant sequence, and the complementary variant splint oligonucleotide can outcompete the wildtype splint oligonucleotide which is not complementary to the variant sequence (e.g., one or more nucleotides) in the gap sequence. The competition among splint oligonucleotides can allow the use of short (e.g., 2 nucleotides) splint oligonucleotides, while achieving specificity of splint oligonucleotide hybridization and/or ligation, for instance, when splint oligonucleotide hybridization and ligation are performed in the same reaction mix and/or the same reaction condition. In some embodiments, using a low hybridization temperature, less denaturation, and/or more co-factors such as Mg²⁺ or other factors that promote hybridization allows the use of shorter splint oligonucleotides.

In some embodiments, upon hybridization to the target nucleic acid molecule, the 5′ terminal nucleotide of the splint oligonucleotide is adjacent to the 3′ terminal nucleotide of the probe or probe set, and the 3′ terminal nucleotide of the splint oligonucleotide is adjacent to the 5′ terminal nucleotide of the probe or probe set. In some embodiments, the 5′ terminal nucleotide of the splint oligonucleotide and the 3′ terminal nucleotide of the probe or probe set are separated by a nick or a gap of one or more nucleotides, e.g., 1, 2, 3, 4, 5, 6, 7, 8, 9, 10, or more nucleotides. In some embodiments, the gap is between about 1 and about 3 nucleotides in length. In some embodiments, the 3′ terminal nucleotide of the splint oligonucleotide and the 5′ terminal nucleotide of the probe or probe set are separated by a nick or a gap of one or more nucleotides, e.g., 1, 2, 3, 4, 5, 6, 7, 8, 9, 10, or more nucleotides. The nick can be ligated using any suitable ligase disclosed herein, and the gap can be filled using any suitable enzyme (e.g., a polymerase) followed by ligation, for example, as described in Section IV-A. In some embodiments, the probe or probe set is circularized using a combination of gap-fill oligonucleotide hybridization and gap-fill polymerization (e.g., primer extension of the 3′ end of the probe or probe set and primer extension of the 3′ end of a gap-fill oligonucleotide) to generate a circularized probe comprising a gap-filled region complementary to the gap sequence.

In some embodiments, the probe or probe set is hybridized to the target nucleic acid, followed by contacting the biological sample with a library of splint oligonucleotides that compete for hybridization to the target nucleic acid (e.g., hybridization to the gap sequence in the target nucleic acid). In some embodiments, the hybridization of a splint oligonucleotide to the target nucleic acid and the ligation of the splint oligonucleotide to the circularizable probe are performed sequentially, e.g., the splint oligonucleotide hybridization is performed in a reaction condition or reaction mix, and the splint oligonucleotide ligation is performed in a different reaction condition or different reaction mix. In some embodiments, the hybridization of a splint oligonucleotide to the target nucleic acid and the ligation of the splint oligonucleotide to the probe or probe set are performed in the same reaction condition or the same reaction mix. In some embodiments, any one or more of the splint oligonucleotides in the library is 2 nucleotides or more in length.

In some embodiments, the probe or probe set and the library of splint oligonucleotides is contacted with the target nucleic acid at the same time, in the same reaction mix or separately. For example, the probe or probe set and the library of splint oligonucleotides is premixed before contacting the biological sample with the mixture. In another example, two separate compositions comprising the probe or probe set and the library of splint oligonucleotides, respectively, is contacted with the biological sample. In some embodiments, the hybridization of a splint oligonucleotide to the target nucleic acid and the ligation of the splint oligonucleotide to the probe or probe set are performed in the same reaction condition or the same reaction mix. In some embodiments, any one or more of the splint oligonucleotides in the library can be 2 nucleotides or more in length.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the splint oligonucleotide comprises a sequence complementary to the gap sequence in the target nucleic acid molecule. In some embodiments, the biological sample is contacted with a library of splint oligonucleotides. In some embodiments, the library comprises at least about 2, at least about 4, at least about 10, at least about 20, at least about 50, at least about 100, or more oligonucleotides of different sequences. In some embodiments, the sequence diversity of the splint oligonucleotides in the library is such that at least or about 80%, at least or about 85%, at least or about 90%, at least or about 95%, or about 100% of the possible variant sequences in the gap sequence of the target nucleic acid in a sample have corresponding splint oligonucleotides in the library, e.g., the splint oligonucleotides comprise sequences that are complementary to the variant sequences in the target nucleic acid.

In some embodiments, the gap filling is performed under conditions permissive for specific hybridization of a splint oligonucleotide to its complementary sequence in the gap sequence in the target nucleic acid molecule, and/or specific hybridization of a probe or probe set to the target nucleic acid molecule. In some embodiments, the probe or probe set comprises hybridization regions that hybridize to the target nucleic acid molecule at sequences flanking the gap sequence (e.g., at constant region sequences on the 5′ the 3′ of the hotspot for mutation), whereas the variant sequences in the gap sequence are complementary to the splint oligonucleotides (e.g., wildtype or mutant) in the library. In some embodiments, the circularized probe is amplified by RCA (, and the RCA product comprises multiple copies of the gap sequence in the target nucleic acid. In some embodiments, a sequence in the gap sequence in the RCA product is determined in situ.

In some embodiments, the splint oligonucleotides is between about 6 and about 24 nucleotides in length. In some embodiments, any one or more of the splint oligonucleotides in the library is about 6, about 8, about 10, about 12, about 14, about 16, about 18, about 20, about 22, or about 24 nucleotides in length. Any two or more of the splint oligonucleotides in the library has the same length or different lengths. In some embodiments, the splint oligonucleotides in the library is of the same length.

In some embodiments, the variant sequence has at least 1, 2, 3, 4, 5, 6, 7, 8, 9, or more phosphodiester bonds from the 3′ or 5′ end of the gap sequence. In some embodiments, the sequence complementary to the variant sequence is at least 1, 2, 3, 4, 5, 6, 7, 8, 9, or more phosphodiester bonds from the 5′ or 3′ end of the splint oligonucleotide. In some embodiments, the variant sequence is at or near the central nucleotide(s) of the gap sequence. In some embodiments, the sequence complementary to the variant sequence is at or near the central nucleotide(s) of the splint oligonucleotide. In some embodiments, the sequence complementary to the variant sequence is no more than 1, no more than 2, no more than 3, no more than 4, no more than 5, or no more than 6 nucleotides from the central nucleotide(s) of the splint oligonucleotide.

In some embodiments, a method disclosed herein comprises hybridizing a gap-fill padlock probe on conserved regions that flank a gap sequence in a nucleic acid target sequence. In some instances, the conserved regions is present in the majority or all RNA transcripts from the same gene (e.g., KRAS), and the gap sequences in the RNA transcripts comprise one or more variant sequences (each of one or more bases), or regions to be interrogated (e.g., one or more SNPs) depending on the particular transcript. In some embodiments, the gap sequences comprise mutation hotspots. In some embodiments, a gap between the arms of a gap-fill padlock probe hybridized to conserved regions in the nucleic acid target are filled using the gap sequence as a template, thereby incorporating sequence information regarding the variant sequence(s) from the nucleic acid target into the gap-filled padlock probe. As used herein, in some embodiments, a gap sequence is an intervening sequence (of one or more bases) between a first target sequence and a second target sequence in a target nucleic acid, and the gap sequence is linked to the first and second target sequences via one or more phosphodiester bonds. In some embodiments, the first and second target sequences are targets of a first probe or probe set, such as arms of a gap-fill padlock probe that do not comprise a variant sequence or an interrogatory region. In some embodiments, the gap sequence comprises one or more variant sequences or regions to be interrogated, whereas the flanking first and second target sequences are constant or invariant among multiple target nucleic acid molecules. In some embodiments, the gap sequence comprises a nucleotide of interest. In some embodiments, the nucleotide of interest is a SNP. In some embodiments, the splint oligonucleotides compete with one another for hybridization to a gap sequence that contains particular variant sequence(s). For instance, in the case of KRAS, mutations occur most frequently in 5 bases in codons 12 and 13. A library of splint oligonucleotides can be designed to cover all possible variants (or any subset thereof) in that region with a length of about 12 bases. In some embodiments, the splint oligonucleotides is between about 6 and about 18 bases. In some embodiments, with short splint oligonucleotides, a single mismatch of one base, especially when the mismatch is in the middle of a short splint oligonucleotide, can reduce the stability of the splint oligonucleotide hybridization and the fully correct matching splint oligonucleotide is favored. Compared to a one-piece circularizable probe strategy where one arm of the probe matches a conserved region in the target sequence and a mismatch on the other arm (e.g., the arm containing an SNP-interrogatory nucleotide) does not de-stabilize the arm matching the conserved region, the splint oligonucleotide approach allows competition among splint oligonucleotides and dissociation of mismatched splint oligonucleotides prior to probe ligation.

In some embodiments, the method further comprises circularizing the gap-filled probe or probe set to generate a circularized probe comprising a gap-filled region complementary to the gap sequence. In some embodiments, a splint oligonucleotide of the library of splint oligonucleotides that is complementary to the gap sequence is ligated to the probe or probe set. In some embodiments, the splint oligonucleotide is ligated to the probe or probe set using the target RNA as a template and a ligase having an RNA-templated DNA or RNA ligase activity. In some embodiments, the ligase is a Chlorella virus DNA ligase (PBCV DNA ligase) or a T4 RNA ligase. In some embodiments, the ligase is a PBCV-1 DNA ligase or a T4 RNA ligase 2. In some embodiments, the extended probe is ligated to the probe or probe set using the target RNA as a template and a ligase having an RNA-templated DNA or RNA ligase activity. In some embodiments, the ligase is a Chlorella virus DNA ligase (PBCV DNA ligase) or a T4 RNA ligase. Optionally, in some embodiments the ligase is a PBCV-1 DNA ligase or a T4 RNA ligase 2. In some instances, gap filling and ligation to circularize the probe or probe set is performed sequentially. In some instances, gap filling and ligation to circularize the probe or probe set is performed in the same reaction.

In some embodiments, the ligation of the splint oligonucleotides to the probes or probe sets (e.g., gap-fill padlock probes) is performed using RNA-templated ligation. In some embodiments, the ligation is performed after hybridization of the splint oligonucleotide library and removing splint oligonucleotides mismatched on target nucleic acids, and the method comprises ligating splint oligonucleotides matched with target nucleic acids to the circularizable probes. In some embodiments, the ligation is performed simultaneously with splint oligonucleotide hybridization, e.g., a ligase is present during splint oligonucleotide hybridization. In some embodiments, the splint oligonucleotide library and a plurality of circularizable probes are contacted with the sample simultaneously or in any order. In some embodiments, the sample is contacted with the splint oligonucleotide library and the plurality of circularizable probes at the same time, and the splint oligonucleotide library and the plurality of circularizable probes can be pre-mixed or not pre-mixed prior to contacting the sample. In some embodiments, the plurality of circularizable probes are hybridized to target nucleic acids in the sample before the splint oligonucleotide library is hybridized and ligated to the circularizable probes. In some embodiments, the splint oligonucleotide library is hybridized to target nucleic acids in the sample before the plurality of circularizable probes are hybridized and ligated to the splint oligonucleotides.

In some embodiments, one or more washes are performed between any of probe or probe set hybridization, splint oligonucleotide hybridization, and ligation. In some embodiments, any one or more of the washes can be stringent so that only completely complementary splint oligonucleotides remain bound to target nucleic acids after the wash(es). In some embodiments, any one or more of the washes can be performed under less than stringent conditions. In some embodiments, any one or more of the washes can be performed under extremely low stringency conditions, low stringency conditions, or medium stringency conditions.

### V. Rolling Circle Amplification (RCA)

In some embodiments, herein is presented a method for detecting a variant sequence of interest in a biological sample, further comprising using a polymerase to amplify the circularized probe to generate a rolling circle amplification product (RCP) comprising multiple copies of the region of interest and the barcode sequence in the biological sample. Following formation of the circularized probe, in some instances, a primer oligonucleotide is added for amplification. In some instances, the primer oligonucleotide is added with the probe or probe set. In some instances, the primer oligonucleotide is added before or after the probe or probe set is contacted with the sample. In some instances, the primer oligonucleotide for amplification of the circularized probe comprises a sequence complementary to a target nucleic acid, as well as a sequence complementary to the probe or probe set that hybridizes to the target nucleic acid. In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some embodiments, a primer oligonucleotide for amplification of the circularized probe comprises a single-stranded nucleic acid sequence having a 3′ end that can be used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. In some embodiments, the primer oligonucleotide is about 6 bases to about 100 bases, such as about 25 bases.

In some embodiments, amplification of the circularized gap-filled first probe or probe set is primed by the target RNA. In some embodiments, the target RNA is cleaved by an enzyme (e.g., RNase H). In some embodiments, the target RNA is cleaved at a position downstream of the first and second target sequences. In some aspects, the methods disclosed herein allow targeting of RNase H activity to a particular region in a target RNA that is adjacent to or overlapping with a target sequence for the probe or probe set (which is be gap-filled and circularizable, e.g., by ligation). For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself is then be used to prime RCA of the circularized probe generated from a circularizable probe or probe set (e.g., target-primed RCA). In some cases, a plurality of nucleic acid oligonucleotides is used to perform target-primed RCA for a plurality of different target RNAs.

In any of the embodiments herein, the biological sample is contacted with the RNase H (and optionally with the nucleic acid oligonucleotide) before or during formation of the circularized gap-filled probe or probe set. In some embodiments, the biological sample is contacted with the oligonucleotide and with the RNase H simultaneously or sequentially (in either order) before contacting the sample with the probe or probe set. In any of the embodiments herein, the biological sample is contacted with the RNase H (and optionally with the nucleic acid oligonucleotide) after formation of the circularized gap-filled probe or probe set. In some embodiments, the probe or probe set hybridizes to the cleaved target RNA, and the cleaved target RNA is used to prime RCA (optionally after gap-fill and ligation reaction(s) to circularize the probe or probe set). In any of the embodiments herein, the RNase H is an RNase H1 and/or an RNAse H2. In some embodiments, RNase inactivating agents or inhibitors is added to the sample after cleaving the target RNA.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a probe or probe set), the circularized probe is rolling-circle amplified to generate a RCA product (e.g., amplicon) containing multiple copies of the sequence of the circularized probe.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Examples of polymerases are described in U.S. Pat. Nos. 8,257,954; 8,133,672; 8,343,746; 8,658,365; 8,921,086; and 9,279,155, each of which is hereby incorporated by reference in its entirety. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some aspects, during the amplification step, modified nucleotides are added to the reaction to incorporate the modified nucleotides in the amplification product (e.g., nanoball). Examples of modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide reacts with an acrylic acid N-hydroxysuccinimide moiety. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on.

In some embodiments, the primer extension reaction mixture comprises a deoxynucleoside triphosphate (dNTP) or derivative, variant, or analogue thereof. In some embodiments, the primer extension reaction mixture can comprise a catalytic cofactor of the polymerase. In any of the embodiments herein, the primer extension reaction mixture can comprise a catalytic di-cation, such as Mg²⁺ and/or Mn²⁺.

In some aspects, the amplification product (e.g., RCA product) is anchored to a polymer matrix. The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some embodiments, the amplification products (e.g., RCA products) are copolymerized and/or covalently attached to the surrounding matrix thereby preserving their spatial relationship and any information inherent thereto. In some embodiments, the RCA products can also be functionalized to form covalent attachment to the matrix preserving their spatial information within the cell thereby providing a subcellular localization distribution pattern. In some embodiments, the provided methods involve embedding RCA products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing or probe hybridization while an existing hydrogel-tissue chemistry method cannot. In some embodiments, amine-modified nucleotides are comprised in the amplification step (e.g., RCA), functionalized with an acrylamide moiety using acrylic acid N-hydroxysuccinimide esters, and copolymerized with acrylamide monomers to form a hydrogel (e.g., for amplification product embedding in the tissue-hydrogel setting).

### VI. Nucleic Acid Analytes and Target Sequences

In some aspects, provided herein are methods and compositions for analysis of target nucleic acids. In some embodiments, the target nucleic acid is targeted by a circularizable probe or probe set (e.g., a gap-fill circularizable probe or probe set such as any of the probe or probe sets described in Section IV). In some embodiments, a rolling circle amplification product is generated, and a sequence in the rolling circle amplification product is detected using a detectably labeled nuclease-deficient Argonaute and guide nucleic acid complex, or a sequence in the rolling circle amplification product is cut using a nuclease-active Argonaute protein and guide nucleic acid.

In some embodiments, the target nucleic acids comprise RNA. In some embodiments, the target nucleic acids comprise DNA. In some embodiments, the target nucleic acids comprise double-stranded DNA. In some embodiments, the target nucleic acids comprise single-stranded DNA. In some embodiments, the target nucleic acids comprise genomic DNA. In some embodiments, the target nucleic acids comprise cDNA. In some embodiments, one or more target nucleic acids each comprises a variant sequence of one or more nucleotides. In some embodiments, one or more target nucleic acids each comprises a single-nucleotide polymorphism (SNP). In some embodiments, one or more target nucleic acids each comprises a single-nucleotide variant (SNV). In some embodiments, one or more target nucleic acids each comprises a single-nucleotide substitution. In some embodiments, one or more target nucleic acids each comprises a point mutation. In some embodiments, one or more target nucleic acids each comprises a single-nucleotide insertion. In some embodiments, one or more target nucleic acids each comprises a single-nucleotide deletion. In any of the embodiments herein, target genomic DNA, target RNA, and/or target cDNA comprising one or more sequence variants at one or more genomic loci can be analyzed as described herein. In some embodiments, target genomic DNA, target RNA, and/or target cDNA comprising one or more single-nucleotide differences (e.g., SNPs, SNVs, point mutations, etc.) at one or more genomic loci can be analyzed, and the identity of one or more single-nucleotide differences can be determined in situ in a sample.

In some embodiments, the target nucleic acid is a cellular nucleic acid analyte or a product thereof. Optionally, in some embodiments, the cellular nucleic acid analyte is an RNA and the product thereof is a cDNA. In some embodiments, the target nucleic acid is associated with a non-nucleic acid analyte. Optionally, in some embodiments, the target nucleic acid is an oligonucleotide reporter in a labeling agent that binds to the analyte. In some embodiments, the target nucleic acid is RNA. Optionally, in some embodiments, the target nucleic acid is mRNA. In some embodiments, the target nucleic acid is a cDNA.

In some embodiments, the target nucleic acid comprises a nucleotide variation, a nucleotide polymorphism, a mutation, a substitution, an insertion, a deletion, a translocation, a duplication, an inversion, and/or a repetitive sequence, in a variant sequence among a plurality of different sequences to be identified in situ in a biological sample or using a spatial assay. In some embodiments, the variant sequence is a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the variant sequence comprises multiple nucleotides, and each nucleotide is independently at the position of an SNV, an SNP, a point mutation, a single nucleotide substitution, a single nucleotide insertion, or a single nucleotide deletion. In some embodiments, the target nucleic acid is an RNA, such as an miRNA or a transcript of an oncogene, a tumor suppressor gene, an immune gene, or an antigen receptor gene.

In some embodiments, the variant sequence and/or the alternative sequence comprises a nucleotide variation, a nucleotide polymorphism, a mutation, a substitution, an insertion, a deletion, a translocation, a duplication, an inversion, and/or a repetitive sequence. In some embodiments, the variant sequence and/or the alternative sequence comprises two or more nucleotide residues. In some embodiments, the variant sequence and/or the alternative sequence is a single nucleotide. In some embodiments, the variant sequence and/or the alternative sequence comprises a single nucleotide variation (SNV), a single nucleotide polymorphism (SNP), a point mutation, a single nucleotide substitution, a single nucleotide insertion, or a single nucleotide deletion. In some embodiments, the variant sequence is the degradation sequence. In some embodiments, the alternative sequence is the degradation sequence. In some embodiments, the degradation sequence is or comprises a barcode sequence. The methods, probes, and kits disclosed herein can be used to detect and analyze a wide variety of different analytes. Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to allow access of one or more reagents (e.g., probes for analyte detection) to the analytes in the cell or cell compartment or organelle.

Analytes of particular interest may include nucleic acid molecules (e.g., cellular nucleic acids), such as DNA (e.g. genomic DNA, cDNA, mitochondrial DNA, plastid DNA, viral DNA, etc.) and RNA (e.g. mRNA, microRNA, rRNA, snRNA, viral RNA, etc.), and synthetic and/or modified nucleic acid molecules (e.g. including nucleic acid domains comprising or consisting of synthetic or modified nucleotides such as LNA, PNA, morpholino, etc.).

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature (e.g., area) of the substrate.

In any embodiment described herein, the analyte can comprise or be associated with a target sequence. In some embodiments, the target nucleic acid and the target sequence therein may be endogenous to the sample, generated in the sample, added to the sample, or associated with an analyte in the sample. In some embodiments, the target sequence is a single-stranded target sequence. In some embodiments, the analytes comprise one or more single-stranded target sequences.

In some embodiments, provided herein are methods for analysis of a target nucleic acid using primer extension to gap-fill a probe or probe set upon its hybridization to a target nucleic acid, and the gap-filled probe or probe set can be circularized by ligation. In some embodiments, the circularized probes are amplified (e.g., using RCA) and the amplicons are detected in situ. In some embodiments, an Argonaute-guide nucleic acid complex (e.g., an Argonaute protein and a guide nucleic acid comprising a seed sequence described herein) is hybridized to an amplicon (e.g., an RCP) at a variant sequence. In some embodiments, the amplicons (of the probe or probe set) and/or the Argonaute-guide nucleic acid complexes targeting various target nucleic acids at variant sequences therein are detected in situ, e.g., using sequential probe hybridization to barcode sequences in the amplicons and/or barcode sequences in the ligation products.

In some embodiments, probes or probe sets that target common regions adjacent to hotspots for mutation are used. In some embodiments, the common regions flank a gap sequence in the target nucleic acid. In some embodiments, the gap sequence comprises one or more hotspots for mutation. In some embodiments, the gap sequence comprises a variant sequence among a plurality of different variant sequences. In some embodiments, gaps in the probes or probe sets upon hybridization to their nucleic acid targets are filled by polymerization. In some embodiments, the gaps are filled by splint ligation, using a library of splint oligonucleotides that are diverse in sequences and comprise a plurality of possible variant sequences (e.g., possible mutations for the hotspots). In some embodiments, the library of splint oligonucleotides is incubated with the sample for hybridization to target nucleic acid molecules, allowing the best matching splint oligonucleotide to outcompete other splint oligonucleotides in the library. In some embodiments, after washing the sample, the best matching splint oligonucleotides are ligated to the probes or probe sets to generate circularized probes, and the circularized probes are amplified.

In some embodiments, the incorporation of variant sequence information into a probe or probe set disclosed herein can be hypothesis-free, requiring no prior knowledge of whether and which particular variant sequences are present in target nucleic acid molecules in a sample. In some embodiments, the gaps in the probes or probe sets hybridized to target nucleic acid molecules can be filled using polymerization (e.g., reverse transcription) templated on the gap sequences, followed by circularization of the extended probes or probe sets (e.g., using a ligase having RNA-templated ligase activity).

In some embodiments, the incorporation of variant sequence information into the probes can take into consideration possible variant sequences covered by the gap sequences. In some embodiments, at least some of the gaps are filled by ligating a library of splint oligonucleotides comprising complementary sequences to the possible variant sequences with the probes or probe sets.

### VII. Samples and Sample Processing

In some embodiments, a sample disclosed herein is derived from any biological sample. In some embodiments, the methods and compositions disclosed herein are used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). In some embodiments, a biological sample is from an organism and comprise one or more other organisms or components therefrom. For example, a mammalian tissue section may comprise a prion, a viroid, a virus, a bacterium, a fungus, or components from other organisms, in addition to mammalian cells and non-cellular tissue components. In some embodiments, subjects from which biological samples are obtained re healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, the biological sample include any number of macromolecules, for example, cellular macromolecules and organelles (e.g., mitochondria and nuclei). In some embodiments, the biological sample comprises nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. In some embodiments, the sample is a fluid sample, such as a blood sample, urine sample, or saliva sample. In some embodiments, the sample comprises a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

In some embodiments, a biological sample is harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section is prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. In some embodiments, the thickness of the tissue section is at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

In some embodiments, multiple sections are obtained from a single biological sample. For example, multiple tissue sections are obtained from a surgical biopsy sample by performing serial sectioning of the biopsy sample using a sectioning blade. In some embodiments, spatial information among the serial sections are preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample is prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than-25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. In some embodiments, prior to analysis, the paraffin-embedding material is removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes as described in Section IV. In some embodiments, one or more post-fixing step is performed after a hybridization complex comprising a probe or probe set and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probe or probe sets) into the sample. If a sample is not permeabilized sufficiently, the transfer of species (such as probe or probe sets) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents are added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. In some embodiments, proteinase K treatment is used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample is embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto is anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel comprises hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221): 543-548, 2015, the entire contents of which are incorporated herein by reference.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, in some embodiments, biological samples are stained using a wide variety of stains and staining techniques. In some embodiments, for example, a sample is stained using any number of stains and/or immunohistochemical reagents. In some embodiments, one or more staining steps are performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain is specific to proteins, phospholipids, DNA (e.g., dsDNA, ssDNA), RNA, an organelle or compartment of the cell. In some embodiments, the sample is contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

In some embodiments, the sample is stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, or Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### VII. Compositions, Kits, and Systems

Provided herein are kits and/or systems, for example comprising one or more oligonucleotides, e.g., any described in Sections I-V, and instructions for performing the methods provided herein. In some embodiments, the kits and/or systems further comprise one or more reagents for performing the methods provided herein. In some embodiments, the kits and/or systems further comprise one or more reagents required for one or more steps comprising hybridization, ligation, extension, amplification, detection, and/or sample preparation as described herein. In some embodiments, the kit and/or system further comprises any one or more of the probe or probe set, the Argonaute-guide nucleic acid complexes, and/or detectably labeled oligonucleotides disclosed herein. In some embodiments, any or all of the oligonucleotides are DNA molecules. In some embodiments, the kit and/or system further comprises an enzyme such as a ligase and/or a polymerase described herein. In some embodiments, the ligase has DNA-splinted DNA ligase activity. In some embodiments, the kit and/or system comprises a polymerase, for instance for performing extension of the primers to incorporate modified nucleotides into cDNA products of transcripts. In some embodiments, the kits may contain reagents for forming a functionalized matrix (e.g., a hydrogel), such as any suitable functional moieties. In some examples, also provided are buffers and reagents for tethering the modified primers, cDNA products, and/or RCA products to the functionalized matrix. The various components of the kit and/or system may be present in separate containers or certain compatible components may be pre-combined into a single container. In some embodiments, the kits and/or systems further contain instructions for using the components of the kit to practice the provided methods.

In some embodiments, provided herein is a kit for analyzing a biological sample, comprising: a) a circularizable probe comprising a first probe region and a second probe region that hybridize to a first target sequence and a second target sequence, respectively, in a target nucleic acid (e.g., RNA) in the biological sample, wherein the first and second target sequences are separated by a gap sequence (e.g., one or more nucleotides) in the target nucleic acid (e.g., RNA), and the gap sequence comprises a variant sequence among a plurality of different variant sequences; b) one or more reagents for circularizing the circularizable probe to generate a circularized probe comprising a gap-filled region complementary to the gap sequence; c) one or more reagents for generating a rolling circle amplification product (RCP) of the circularized probe, wherein the RCP comprises multiple copies of the gap sequence; and/or d) one or more Argonaute-guide nucleic acid complexes comprising a slicer-dead Argonaute protein, a guide nucleic acid comprising a seed region complementary to the variant sequence of the plurality of variant sequences, and a detectable label corresponding to the variant sequence of the plurality of variant sequences such that the Argonaute-guide nucleic acid complex can bind to the RCP at the variant sequence of the plurality of variant sequences and be detected in situ. In some embodiments, the one or more Argonaute-guide nucleic acid complexes comprises a slicer-dead Argonaute protein preloaded with a guide nucleic acid. In some embodiments, the kit comprises a mixture of preloaded Argonaute-guide nucleic acid complexes. In some embodiments, provided herein is a system comprising the kit and the biological sample.

In some embodiments, provided herein is a kit for analyzing a biological sample, comprising: a) a circularizable probe comprising a first probe region and a second probe region that hybridize to a first target sequence and a second target sequence, respectively, in a target nucleic acid (e.g., RNA) in the biological sample, wherein the first and second target sequences are separated by a gap sequence in the target nucleic acid (e.g., RNA), and the gap sequence comprises a variant sequence among a plurality of different variant sequences; b) one or more reagents for circularizing the circularizable probe to generate a circularized probe comprising a gap-filled region complementary to the gap sequence; c) one or more reagents for generating a rolling circle amplification product (RCP) of the circularized probe, wherein the RCP comprises multiple copies of the gap sequence; and/or d) one or more Argonaute-guide nucleic acid complexes comprising a slicer-active Argonaute protein capable of cutting the RCP at a variant sequence among a plurality of variant sequences (e.g., either a variant sequence of interest or an alternative variant sequence). In some embodiments, provided herein is a system comprising the kit and the biological sample.

In some embodiments, a kit disclosed herein comprises a pool of detection oligonucleotides each comprising a detectable label. In some embodiments, the biological sample is imaged to detect signals associated with the detectable labels at locations in the biological sample, thereby detecting one or more of the plurality of different variant sequences in the biological sample. In some embodiments, the one or more of the plurality of different variant sequences are identified (e.g., the identity of an SNP or point mutation is revealed) in the biological sample, based on the signals detected at the locations. In some embodiments, provided herein is a system comprising the kit and the biological sample.

In some embodiments, a kit disclosed herein comprises a) a circularizable probe comprising a first probe region and a second probe region that hybridize to a first target sequence and a second target sequence, respectively, in a target nucleic acid in the biological sample, wherein the first and second target sequences are separated by a gap sequence in the target nucleic acid, and wherein the gap sequence comprises a variant sequence among a plurality of different variant sequences; b) a library of splint oligonucleotides, wherein each splint oligonucleotide comprises: i) ligatable ends; and ii) a hybridization region complementary to one of the plurality of different variant sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence. In some embodiments, each splint oligonucleotide of the library comprises a phosphate group on the 5′-end available for ligation; and c) one or more Argonaute-guide nucleic acid complexes comprising a slicer-dead Argonaute protein, a guide nucleic acid comprising a seed region complementary to the variant sequence of the plurality of variant sequences, and a detectable label corresponding to the variant sequence of the plurality of variant sequences such that the Argonaute-guide nucleic acid complex can bind to the RCP at the variant sequence of the plurality of variant sequences and be detected in situ. In some embodiments, provided herein is a system comprising the kit and the biological sample. In some embodiments, a system or kit comprises a plurality of different guide nucleic acids comprising seed sequences complementary to a plurality of different variant sequences.

In some embodiments, a kit disclosed herein comprises a) a circularizable probe comprising a first probe region and a second probe region that hybridize to a first target sequence and a second target sequence, respectively, in a target nucleic acid in the biological sample, wherein the first and second target sequences are separated by a gap sequence in the target nucleic acid, and wherein the gap sequence comprises a variant sequence among a plurality of different variant sequences; b) a library of splint oligonucleotides, wherein each splint oligonucleotide comprises: i) ligatable ends; and ii) a hybridization region complementary to one of the plurality of different variant sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence. In some embodiments, each splint oligonucleotide of the library comprises a phosphate group on the 5′-end available for ligation; and c) one or more Argonaute-guide nucleic acid complexes comprising a slicer-active Argonaute protein capable of cutting the RCP at a variant sequence among a plurality of variant sequences (e.g., either a variant sequence of interest or an alternative variant sequence). In some embodiments, provided herein is a system comprising the kit and the biological sample.

The biological sample, the circularizable probe (e.g., in a composition comprising a plurality of circularizable probes), and the library of splint oligonucleotides can be contacted with one another in any order. For instance, the circularizable probe and the library of splint oligonucleotides can be pre-mixed prior to contacting the biological sample with the mixture. In other examples, the biological sample can be contacted with the circularizable probe and then with the library of splint oligonucleotides. In some examples, the biological sample are contacted with the library of splint oligonucleotides and then with the circularizable probe. In still other examples, the circularizable probe and the library of splint oligonucleotides are provided in separate compositions which are contacted with the biological sample simultaneously. In some embodiments, the method comprises generating a rolling circle amplification product (RCP) of the circularized probe in the biological sample, wherein the RCP comprises multiple copies of the gap sequence. In some embodiments, the method comprises detecting a sequence comprising the variant sequence in the gap sequence of the RCP at a location in the biological sample, thereby detecting the target nucleic acid comprising the variant sequence at the location in the biological sample. The method may but does not need to comprise detecting a barcode sequence in the RCP. In some embodiments, provided herein is a system comprising the kit and the biological sample.

In some embodiments, a kit disclosed herein comprises a probe or probe set comprising a first probe region and a second probe region that hybridize to a first target sequence and a second target sequence, respectively, in a target RNA in the biological sample, wherein the first and second target sequences are separated by a gap sequence in the target RNA, and wherein the gap sequence comprises a variant sequence among a plurality of different variant sequences. In some embodiments, a kit disclosed herein comprises reagents for circularizing the probe or probe set to generate a circularized probe comprising a gap-filled region complementary to the gap sequence. In some embodiments, a kit disclosed herein comprises reagents for generating a rolling circle amplification product (RCP) of the circularized probe in the biological sample, wherein the RCP comprises multiple copies of the gap sequence. In some embodiments, provided herein is a system comprising the kit and the biological sample.

In some aspects, a kit disclosed herein comprises an Argonaute-guide nucleic acid complex comprising an Argonaute protein and a guide nucleic acid of a plurality of guide nucleic acids comprising a seed region complementary to a variant sequence in the RCP. In some embodiments, a kit disclosed herein comprises reagents for detecting the variant sequence in the gap sequence of the RCP at a location in the biological sample, thereby detecting the target RNA comprising the variant sequence at the location in the biological sample. In some embodiments, provided herein is a system comprising the kit and the biological sample.

In some embodiments, a kit disclosed herein comprises: a probe or probe set, and a guide nucleic acid capable of complexing with an Argonaute protein. In some embodiments, the kit additionally comprises the Argonaute protein. In some embodiments, the Argonaute protein is provided separately. In some embodiments, the Argonaute protein is provided by the user. In some embodiments, the Argonaute protein is provided by a third-party supplier.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as detectably labeled oligonucleotides or detectable labels. In some embodiments, the kits optionally contain other components, for example nucleic acid primers, enzymes and reagents, buffers, nucleotides, modified nucleotides, reagents for additional assays. In some embodiments, provided herein are systems comprising kits disclosed herein, and the biological sample.

In some embodiments, the invention is a kit, comprising: a probe or probe set, an Argonaute protein, and a guide nucleic acid capable of complexing with the Argonaute protein. In some embodiments, the probe or probe set comprises a first probe region and a second probe region that hybridize to a first target sequence and a second target sequence, respectively, in a target nucleic acid in the biological sample. In some embodiments, the Argonaute is nuclease inactive. In some embodiments, the nuclease inactive Argonaute and guide nucleic acid bind to the target nucleic acid and are detected. In some embodiments, the Argonaute is nuclease active. In some embodiments, the nuclease active Argonaute cuts the RCP at a target sequence in the target nucleic acid. In some embodiments, provided herein is a system comprising a kit and the biological sample.

In some embodiments, the Argonaute-guide nucleic acid complex of any of the above kits or systems is detectably labeled. In some embodiments, the Argonaute-guide nucleic acid complex comprises a detectable moiety (e.g., a fluorescent label) that allows for it to be detected in situ at a location in the biological sample when bound to an RCP generated in the biological sample at a variant sequence. In some embodiments, the Argonaute protein of the Argonaute-guide nucleic acid complex is detectably labeled. In some embodiments, the guide nucleic acid of the Argonaute guide nucleic acid complex comprises a detectable label, optionally wherein the detectable label is attached to the 3′ tail region.

### IX. Applications

In some aspects, the provided embodiments are applied in an in situ method of analyzing nucleic acid sequences in intact tissues or samples in which the spatial information has been preserved (e.g., a cleared biological sample embedded in a matrix). In some aspects, the embodiments are applied in an imaging or detection method for multiplexed nucleic acid analysis. In some aspects, the provided embodiments are used to identify or detect mutations in a target nucleic acid. In some aspects, the target nucleic acid is an RNA. In some embodiments, the target nucleic acid is an mRNA. In some aspects, the target nucleic acid is a DNA. In some embodiments, the target nucleic acid is genomic DNA. In some aspects, the target nucleic acid is cDNA. In some aspects, the provided embodiments can be used to crosslink the RCA products via modified nucleotides, e.g., to a matrix, to increase the stability of the circularizable probe or probe set, or the RCA products in situ.

In some aspects, the embodiments are applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples. In some aspects, the embodiments can be applied to visualize the distribution of genetically encoded markers in whole tissue at subcellular resolution.

In some aspects, Argonaute-mediated obliteration of a variant sequence among a plurality of variant sequences in a biological sample decreases optical crowding of fluorescent detection of variant sequences in situ to allow for improved sample decoding at a location in a biological sample, for example, as shown in FIG. 6A and FIG. 6B. In some embodiments, the optical crowding is a phenomenon wherein detectable fluorescent labels are present in a tissue sample at a density that is difficult to visually resolve. For example, if too many detectable labels are present in the same voxel, the detectable labels cannot all be accurately spatially resolved and decoded, as shown in FIG. 6A. Optical crowding results in both a limit to the number of molecules that can be detected, as well as a limit to the detection efficiency and accuracy within a given tissue. Selectively obliterating a defined variant sequence of a plurality of variant sequences may improve signal detection and decoding, as shown in FIG. 6B. In some aspects, selective obliteration of a variant sequence within a biological sample has widespread applications for sample improving and processing. In certain embodiments, selective obliteration of a wild-type variant increases the sensitivity of an assay for detecting one or more mutant variants at a location in the biological sample. As another example, in some cases, selective obliteration of a variant sequence associated with contamination or experimental artifacts is useful for optimizing a sample for downstream workflows, or for cleaning poor quality tissue samples. In some embodiments, Argonaute-mediated obliteration of variant sequences may have utility for rescuing biological samples of poor quality such as those obtained under suboptimal conditions.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some embodiments, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The disclosures of all publications, patents, and patent applications referred to herein are each hereby incorporated by reference in their entireties. To the extent that any reference incorporated by reference conflicts with the instant disclosure, the instant disclosure shall control.

### EXAMPLES

The examples are included for illustrative purposes only and are not intended to limit the scope of the present disclosure.

### Example 1: Detecting a Single Nucleotide Variant of Interest In Situ

This example describes a workflow wherein a circularizable probe that hybridizes to a target nucleic acid comprising a single nucleotide variant sequence (e.g., a mutant sequence or a wildtype sequence) is gap-filled and used to generate RCPs comprising copies of the single nucleotide variant sequence, and the single nucleotide variations in the RCPs are then discriminated by contacting the RCPs with a plurality of Argonaute-guide nucleic acid complexes to bind at the variant sequence or a complement thereof.

A tissue sample comprising a target nucleic acid (e.g., mRNA) comprising a single nucleotide variant (SNV) of interest (e.g., a single-nucleotide mutant sequence of interest) is sectioned and the tissue sections are mounted on a slide, fixed (e.g., by incubating in paraformaldehyde (PFA)), washed, and permeabilized (e.g., using Triton-X). After permeabilization, the tissue sections are washed, dehydrated, and rehydrated.

A gap-fill circularizable probe or probe set (e.g., a gap-fill padlock probe) is designed to hybridize to target regions flanking the single nucleotide variant sequence, which may comprise a single nucleotide variant of interest (e.g., a mutant SNV) or an alternative single nucleotide variant (e.g., a wildtype SNV). In some embodiments, the gap-fill circularizable probe additionally comprises an optional barcode region, as shown in FIG. 2. An RNA polymerase enzyme is used to fill the gap in the circularizable probe hybridized to a wildtype or mutant variant transcript, respectively, with one or more nucleotides comprising a complementary sequence to the gap sequence comprising the single nucleotide variant sequence. After hybridization and extension by the polymerase, an extended circularizable probe is generated comprising nucleotides complementary to the gap sequence on the 3′ end or the 5′ end. After gap-filling with RNA polymerase, the tissue sections comprising an extended probe is washed and incubated with a ligase (e.g., a SplintR® ligase or T4 RNA ligase 2) in a ligation buffer to form a circularized probe. Alternatively, the tissue sample is contacted with a gap-fill splint, which is ligated with the gap-fill circularizable probe or probe set to form a circularized probe.

For RCA, the tissue sections are washed and then incubated in an RCA reaction mixture (containing Phi29 reaction buffer, dNTPs, Phi29 polymerase) to generate RCPs containing the wildtype or mutant sequence, such as a single nucleotide variant of interest or an alternate single nucleotide variant.

As shown in FIG. 3B, guide nucleic acids of the Argonaute-guide nucleic acid complexes are designed to bind to the RCPs, and the guide nucleic acids comprise a 5′ seed region for discriminating a single nucleotide variant of interest from an alternative single nucleotide variant via binding of an Argonaute-guide nucleic acid complex. The tissue sections containing RCPs are incubated with the Argonaute-guide nucleic acid complexes in a buffer comprising Mg²⁺ to allow binding of Argonaute-guide nucleic acid complexes to the RCPs at the single nucleotide variant sequence (e.g., either the single nucleotide variant of interest or the alternative single nucleotide variant) complementary to the seed region of the guide nucleic acids.

The Argonaute-guide nucleic acid complexes are detectably labeled with a fluorescent moiety that corresponds to the specific seed sequence designed to bind to a region of the RCP comprising either the single nucleotide variant of interest or the alternative single nucleotide variant, allowing for detection of the single nucleotide variant sequence at a location in the biological sample. A first set of Argonaute-guide nucleic acids comprise guide nucleic acids with a seed region for binding a variant sequence of interest (e.g., a mutant sequence) and a first detectable label (e.g., a green fluorescent moiety such as eGFP) for detecting the variant sequence of interest. A second set of Argonaute-guide nucleic acids comprise guide nucleic acids with a seed region for binding the alternate variant sequence (e.g., a wildtype sequence) and a second detectable label (e.g., a red fluorescent moiety such as mCherry) for detecting the alternative single nucleotide variant sequence (e.g., a wildtype SNV). As shown in FIG. 2, the Argonaute-guide nucleic acid complexes comprising detectable fluorescent labels bind to the RCPs at the variant sequence complementary to the seed sequence of the guide nucleic acid and are subsequently detected in situ. The tissue sections are washed, stained with DAPI, and mounted in a mounting medium for imaging using fluorescent microscopy to detect the fluorescent moieties corresponding to the single nucleotide variant sequence of interest and/or the alternate single nucleotide variant sequence (e.g., the mutant SNV and/or the wildtype SNV).

The tissue sample is imaged using fluorescent microscopy to detect the fluorescent moieties of the guide nucleic acids targeting the variant sequence of interest (e.g., a first fluorescent signal associated with a mutant SNV) and/or the fluorescent moieties of the guide nucleic acids targeting the alternate variant sequence (e.g., a second fluorescent signal associated with a wildtype SNV).

### Example 2: Detecting Heterozygous and Homozygous Single-Nucleotide Variants In Situ Using Argonaute-Mediated Obliteration

This example describes a workflow comprising first detecting a sequence of interest (e.g., a SNP) in an RCP, followed by using slicer-active Argonaute-guide nucleic acid complexes to cleave RCPs comprising the sequence of interest, and then detecting RCPs that are not cleaved. An example schematic of the method described in Example 2 is provided in FIG. 4A. This workflow can be used to identify homozygous and heterozygous variant alleles of interest at a location in the biological sample, as shown in an example decoding scheme in FIG. 4B.

Rolling circle amplification products (RCPs) comprising copies of a variant sequence and copies of a barcode sequence are generated in a biological sample as described in Example 1. For RCA, the tissue sections are washed and then incubated in an RCA reaction mixture (containing Phi29 reaction buffer, dNTPs, Phi29 polymerase) to generate RCPs containing the single nucleotide variant of interest and/or the alternative single nucleotide variant.

A first round of detectably labeled probes comprising a barcode region complementary to a barcode sequence in the RCP and a fluorescent moiety are then contacted with the RCPs. The first round of detectably labeled probes hybridize to the RCPs at the barcode sequence in the RCPs. The biological sample comprising RCPs with bound detectably labeled probes is then imaged in a first round of imaging with fluorescent microscopy to detect the fluorescent moieties of the first round of detectably labeled probes at a location in the biological sample. Following the first round of imaging, the biological sample is washed to remove the first round of detectably labeled probes from the RCPs.

After washing, the biological sample is then contacted with an Argonaute-guide nucleic acid complex comprising a nuclease-active Argonaute protein (e.g., a tTAgo Argonaute protein with slicer activity) and a guide nucleic acid specifically engineered to target the single nucleotide variant sequence of interest. The Argonaute and guide nucleic acid are incubated with the RCPs for two hours in a buffer comprising Mg²⁺ cations to allow the cutting of the RCP at or near the single nucleotide variant sequence of interest by the Argonaute-guide nucleic acid complex. The cutting of the RCPs generates cut RCP fragments that are washed and removed from the biological sample.

A second round of detectably labeled probes, again comprising a barcode region complementary to a barcode sequence in the RCP and a fluorescent moiety, are contacted with the RCPs. The second round of detectably labeled probes are contacted with the RCPs, wherein the barcode region of the detectably labeled probes hybridizes to the barcode sequence in the RCPs. A second round of imaging (e.g., fluorescent microscopy imaging) is performed on the biological sample comprising RCPs with a second round of bound detectably labeled probes to detect the fluorescent moieties of the second round of detectably labeled probes at a location in the biological sample.

The fluorescent signal from the biological sample from the first round of imaging and the fluorescent signal from the second round of imaging are compared to determine the relative change in fluorescence at a location in the biological sample between the first round of imaging and the second round of imaging. Signals are decoded using a decoding scheme as shown, for example, in FIG. 4B. A decrease in fluorescence at a location in the biological sample indicates Argonaute-mediated cutting of the mutant sequence at the location in the biological sample, which indicates the presence of the mutant sequence at the location in the biological sample. No change in fluorescence at a location in the biological sample indicates no Argonaute-mediated cutting of the mutant sequence at the location in the biological sample, which indicates the absence of the mutant sequence at the location in the biological sample. Fluorescence at a location in the biological sample that does not decrease in the second round of imaging indicates the presence of the homozygous wildtype allele sequence at the location in the biological sample. Fluorescence at a location in the biological sample that decreases by about 50% (e.g., about half of the fluorescent puncta at a location from a first round of imaging are not present in a second of imaging) indicates the presence of a heterozygous allele with one copy of the wildtype variant and one copy of the variant allele of interest at a location in the tissue.

### Example 3: Detecting a Variant of Interest In Situ Using Argonaute-Mediated Obliteration of an Alternative Variant

This example illustrates the use of a slicer-active Argonaute-guide nucleic acid complex to cut an alternative variant sequence in an RCP generated in a biological sample, and then detecting a variant sequence of interest at a location in the biological sample using detectably labeled probes.

FIG. 5 provides a schematic illustration of the workflow. Rolling circle amplification of a variant sequence (e.g., a variant sequence that comprises a mutant or a wildtype SNV) is performed with barcoded gap-fill circularizable probes to generate RCPs comprising multiple copies of the variant sequence. A slicer-active Argonaute-guide nucleic acid is used to cut the RCP at or near an alternative variant sequence (e.g., the wildtype SNV), generating cut RCPs that can be washed from the biological sample. Next, the method comprises detecting the presence or absence of the barcode at a location in the biological sample. The remaining RCPs comprising the barcode sequence correspond to the variant sequence of interest (e.g., the mutant SNV). In some embodiments, at or near may mean that the cutting occurs within about 12 or fewer nucleotides, within about 10 or fewer nucleotides, within about 8 or fewer nucleotides, within about 6 or fewer nucleotides, within about 4 or fewer nucleotides, within about 2 or fewer nucleotides, or within about 1 or fewer nucleotides, of the alternative variant sequence (e.g., the wildtype SNV).

RCPs comprising a single nucleotide variant sequence are generated using barcoded gap-fill padlock probes as described in Example 1. An Argonaute-guide nucleic acid complex comprising a slicer-active tTAgo Argonaute protein and a guide nucleic acid with a seed region comprising an interrogatory nucleotide complementary to the alternative variant nucleotide is contacted with the RCPs and cuts the RCPs at or near the alternative variant nucleotide. The Argonaute-guide nucleic acid complexes are incubated with the RCPs in the cutting buffer comprising Mg²⁺ cations allowing for cutting of the RCPs by the Argonaute proteins. Cutting of the RCPs with the TtAgo-guide nucleic acid complex generates cut RCP fragments, which are washed and removed from the biological sample. Detectably labeled probes, comprising a detectable moiety (e.g., a fluorescent label) and a complement of the barcode region in the RCPs, are then contacted with the biological sample. The biological sample and bound detectably labeled probes are imaged, wherein the presence of a detectably labeled probe at a location in the biological sample indicates the presence of the variant nucleotide of interest at the location in the biological sample.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method, comprising
(a) contacting the biological sample with a probe or probe set,
wherein the probe or probe set comprises a first probe region and a second probe region that hybridize to a first target sequence and a second target sequence, respectively, in a target nucleic acid in the biological sample,
wherein the first and second target sequences flank a gap sequence in the target nucleic acid, and
wherein the gap sequence comprises a variant sequence;
(b) performing a gap-fill reaction on the probe or probe set to generate a gap-filled probe or probe set and circularizing the gap-filled probe or probe set;
(c) using a polymerase to amplify the circularized the gap-filled probe or probe set to generate a rolling circle amplification product (RCP) comprising multiple copies of the variant sequence in the biological sample;
(d) contacting the biological sample with a nuclease-deficient Argonaute protein and a guide nucleic acid, wherein the guide nucleic acid comprises a sequence complementary to the variant sequence in the RCP, wherein the Argonaute protein and the guide nucleic acid form a complex with the RCP; and
(e) detecting the complex formed between the Argonaute protein, the guide nucleic acid, and the RCP in the biological sample.

2. The method of claim 1, wherein the Argonaute protein is an RNA-guided Argonaute, and the guide nucleic acid is an RNA molecule.

3. (canceled)

4. The method of claim 1, wherein the Argonaute protein is a DNA-guided Argonaute, and the guide nucleic acid is a DNA molecule.

5-7. (canceled)

8. The method of claim 1, wherein the nuclease-deficient Argonaute protein is a Drosophila Argonaute protein or a derivative or variant thereof.

9-10. (canceled)

11. The method of claim 1, wherein the guide nucleic acid and the Argonaute protein are bound in a pre-formed complex before contacting the biological sample.

12-14. (canceled)

15. The method of claim 1, wherein the nuclease-deficient Argonaute protein is labeled with a detectable moiety, optionally wherein the detectable moiety is a fluorescent dye.

16. The method of claim 1, wherein the guide nucleic acid is labeled with a detectable moiety, optionally wherein the detectable moiety is a fluorescent dye.

17. The method of claim 1, wherein the guide nucleic acid comprises a 3′ tail sequence, and wherein the method comprises contacting the biological sample with a detectably labeled probe that binds directly or indirectly to the 3′ tail sequence, and wherein detecting the complex formed between the Argonaute protein, the guide nucleic acid, and the RCP in the biological sample comprises detecting the complex comprising the detectably labeled probe bound directly or indirectly to the guide nucleic acid.

18. (canceled)

19. The method of claim 1, wherein the contacting in (e) comprises contacting the biological sample with a plurality of different guide nucleic acids comprising seed sequences complementary to a plurality of different variant sequences.

20. The method of claim 1, wherein performing the gap-fill reaction comprises contacting the biological sample with a library of splint oligonucleotides, wherein each splint oligonucleotide comprises:
(i) ligatable ends; and
(ii) a hybridization region complementary to one of a plurality of different sequences, wherein a splint oligonucleotide of the library of splint oligonucleotides that is complementary to the gap sequence is ligated to the probe or probe set.

21. The method of claim 20, wherein the splint oligonucleotide comprises a 3′ hydroxyl group and a 5′ phosphate group, optionally wherein the splint eligenucleotide comprises one or more ribonucleotide residues at and/or near its 3′ end and/or a 5′ flap configured to be cleaved by a structure specific endonuclease.

22-27. (canceled)

28. The method of claim 20, wherein the variant sequence comprises a single nucleotide variation (SNV), a single nucleotide polymorphism (SNP), a point mutation, a single nucleotide substitution, a single nucleotide insertion, or a single nucleotide deletion.

29. The method of claim 20, wherein the target nucleic acid is a target RNA, and the splint oligonucleotide is ligated to the probe or probe set using the target RNA as a template and a ligase having an RNA-templated DNA or RNA ligase activity.

30-33. (canceled)

34. (canceled)

35. The method of claim 1, wherein the target nucleic acid is a target RNA, and performing the gap-fill reaction comprises using a gap-fill polymerase to extend an end of the probe or probe set using the target RNA as a template to generate an extended probe, wherein the extended probe is ligated to another end of the probe or probe set.

36-37. (canceled)

38. The method of claim 35, wherein the extended probe is ligated to the probe or probe set using the target RNA as a template and a ligase having an RNA-templated DNA or RNA ligase activity.

39-44. (canceled)

45. The method of claim 1, wherein the guide nucleic acid comprises a guide sequence complementary to a sequence of the RCP comprising the variant sequence, wherein the guide sequence is between about 14 and 20 nucleotides in length, optionally wherein the guide nucleic acid is between about 16 and 20 nucleotides in length.

46-92. (canceled)

93. The method of claim 1, wherein the target nucleic acid is RNA, optionally wherein the target nucleic acid is-mRNA.

94. The method of claim 1, wherein the target nucleic acid is a cDNA.

95. The method of claim 1, wherein the biological sample is a cell sample or a tissue section.

96-103. (canceled)

104. A system, comprising:
a biological sample;
a probe or probe set, wherein the probe or probe set comprises a first probe region and a second probe region that hybridize to a first target sequence and a second target sequence, respectively, in a target nucleic acid in the biological sample,
wherein the first and second target sequences flank a gap sequence in the target nucleic acid, and wherein the gap sequence comprises a variant sequence;
an Argonaute protein; and
a guide nucleic acid capable of complexing with the Argonaute protein, wherein a seed region of the guide nucleic acid is complementary to the variant sequence.

105-108. (canceled)
